(19) United States
(12) Patent Application Publication
(10) Pub. No.: US 2024/0139233 A1
(43) Pub. Date: May 2, 2024

(54) ACTIVE AGENT-RELEASING HYALURONIC ACID VEHICLE

(71) Applicant: Ariel Scientific Innovations Ltd., Ariel (IL)

(72) Inventors: Gary GELLERMAN, Ariel (IL); Andrii BAZYLEVICH, Ariel (IL)

(73) Assignee: Ariel Scientific Innovations Ltd., Ariel (IL)

(21) Appl. No.: 18/275,622

(22) PCT Filed: Feb. 3, 2022
(86) PCT No.: PCT/IL2022/050148

§ 371 Date: Aug. 3, 2023

Related U.S. Application Data
(60) Provisional application No. 63/145,586, filed on Feb. 4, 2021.

(51) Int. Cl. A61K 31/728; A61K 8/73; A61K 47/61; A61Q 19/00

(52) U.S. Cl. CPC A61K 31/728; A61K 8/735; A61K 47/61; A61Q 19/00

(57) ABSTRACT

A molecular structure comprising a hyaluronic acid moiety or a crosslinked form thereof, and a plurality of controllably released bioactive agents attached thereto is provided herein, as well as processes for obtained the same and methods of treating a skin conditions using the same.

### RELATED APPLICATION

This application claims the benefit of priority under 35 USC § 119(e) of U.S. Provisional Patent Application No. 63/145,586, filed 4 Feb. 2021, the contents of which are incorporated herein by reference in their entirety.

### FIELD AND BACKGROUND OF THE INVENTION

The present invention, in some embodiments thereof, relates to cosmetics, and more particularly, but not exclusively, to a molecular structure based on hyaluronic acid that acts as a multiple-active agent delivery vehicle, and to uses thereof.

Hyaluronic acid (HA), a naturally-occurring glycosaminoglycan (GAG), plays a key role in healing various skin conditions. HA has a range of naturally occurring molecular sizes from 100 to 10,000,000 Da. HA is implicated in water homeostasis of tissues, in the regulation of permeability of other substances by steric exclusion phenomena, and in the lubrication of joints. HA also binds specifically to proteins in the extracellular matrix, on the cell surface, and within the cells cytosol, thereby having a role in cartilage matrix stabilization, cell motility, growth factor action, morphogenesis and embryonic development and inflammation. Unmodified HA has many important application in drug delivery and surgery. For example, it is used as an adjuvant for ophthalmic drug delivery. In addition, HA has important application in the fields of visco-surgery, visco-supplementation and wound healing. HA is also a building-block for biocompatible and biodegradables polymers with application in drug delivery, tissue engineering and visco-supplementation.

Hydrogels are formed by crosslinked polymers and are able to absorb high quantity of water without being dissolved. HA hydrogels are physically or covalently cross-linked HA gel. HA molecules are generally functionalized to allow reaction with a cross linker. Crosslinked HA hydrogels for example have been prepared by crosslinking with molecules such as di-epoxy-butane, ethylene-glycol di-glycidyl-ether (EGDGE), 1,4-butanediol diglycidyl ether (BDDE) or poly-glycol diglycidyl-ether (PEGDE). HA hydrogels have been used for several application including drug delivery applications. They are able to provide sustained, local delivery of a variety of therapeutic agent. Use of HA as a scaffold material in hydrogel has been pursued due to the biocompatibility, low toxicity, lack of immune response and biodegradability of HA hydrogel. HA hydrogels have been used for several application including drug delivery applications. They are able to provide sustained, local delivery of a variety of therapeutic agent. Use of HA as a scaffold material in hydrogel has been pursued due to the biocompatibility, low toxicity, lack of immune response and biodegradability of HA hydrogel.

Although HA hydrogels have been studied for drug delivery applications, the delivery rates are difficult to control. If a hydrophilic drug is incorporated into the hydrogel, the incorporation is easy (large amounts can be loaded), but release is also rapid. On the other hand, it is difficult to get large amounts of hydrophobic drugs into such hydrogels, for solubility reasons. Any undissolved drug will migrate to the surface of the hydrogel and release in a burst (within a day or two).

U.S. Pat. No. 9,987,367 relates to hyaluronic acid (HA) hydrogels comprising vesicles loaded with a drug or a protein or a nucleic acid. The HA hydrogels are said to provide sustain release formulations that are useful for several clinical and surgical applications, including but not limited to ophthalmology (e.g. glaucoma, corneal, ocular inflammatory, vitreoretinal and medical retinal diseases) and dermatological conditions.

### SUMMARY OF THE INVENTION

The present disclosure provides a genus of drug-delivering molecular structures based on linear and/or branched/dendrimeric strings of releasable bioactive compounds tethered to hyaluronic acid (HA) strands. The genus includes single-stranded HA as well as double-stranded and multi-stranded HA stranded, crosslinked by strings of releasable bioactive compounds.

Thus, according to an aspect of some embodiments of the present invention, there is provided a molecular structure represented by Formula I:

HA [symbol] L₁-D₁-L₂-D₂ . . . Lₙ-Dₙ]ₘ Formula I
wherein:
HA is a first hyaluronic acid moiety;
L₁, L₂ . . . and Lₙ are each independently a biocleavable linking moiety;
D₁, D₂ . . . and Dₙ are each independently a bioactive agent moiety;
n is an integer equal or larger than 2; and
m is a positive number equal or larger than 1.

In some embodiments, each of the D₁, D₂ . . . and Dₙ is a moiety of the same bioactive agent.

In some embodiments, each of the D₁, D₂ . . . and Dₙ is a moiety of a different bioactive agent.

In some embodiments, each of the L₁, L₂ . . . and Lₙ is the same biocleavable linking moiety.

In some embodiments, each of the L₁, L₂ . . . and Lₙ is a different biocleavable linking moiety.

In some embodiments, each of the L₁, L₂ . . . and Lₙ is characterized by a different biocleavage condition.

In some embodiments, the molecular structure further includes a second hyaluronic acid moiety attached to the structure via Lₙ.

In some embodiments, the molecular structure having more than one HA strand can be represented by Formula II:

HA₁[-L₁-D₁-L₂-D₂ . . . Lₙ-Dₙ-Lₙ₊₁-HA₂]ₘ Formula II
wherein:
HA₁ is the first hyaluronic acid moiety; and
Lₙ₊₁ is a biocleavable linking moiety;
HA₂ is the second hyaluronic acid moiety.

In some embodiments, the bioactive agent is selected from the group consisting of tranexamic acid, kojic acid, cysteamine, cystamine, azelaic acid, hydroquinone, mequinol, flutamide, CBD, arbutin, vitamin A, vitamin C, vitamin E, ellagic acid, glutathione, biotin, mandelic acid, an alpha-hydroxy acid, tretinoin, an alpha-lipoic acid, glutathione, salicylic acid and fluocinolone.

In some embodiments, the biocleavable linking moiety is selected from the group consisting of —O—, —S—, —S—S—, —Se—, —Se—Se—, —NH—NH—, —NH—, —NH—(CH₂)₂—NH—, —O—CH₂—O—, —O—(CH₂)₂—O—, —(C═O)—NH—, —(C═O)—O—, —(C═O)—S—, —C═NH)—NH—, —(O═O)—NH—, —P(OH)(═O)—NH— and —NH—NH—(C═O)—NH—.

In some embodiments, at least one functional group in the HA exhibits at least one functional group modification moiety.

In some embodiments, at least one of D₁, D₂ . . . and Dₙ is a precursor of a bioactive agent.

In some embodiments, the molecular structure is represented by any one of the structures demonstrated in Examples 1-19 presented below.

According to another aspect of some embodiments of the present invention, there is provided a cosmetic composition that includes one or more of the molecular structure presented herein as an active ingredient, and a cosmetically acceptable carrier.

In some embodiments, the cosmetic composition is packaged in a packaging material and identified in print, in or on the packaging material, for use in the treatment of a skin condition.

According to another aspect of some embodiments of the present invention, there is provided a use of the molecular structure provided herein, in the preparation of a cosmetic composition.

In some embodiments, the cosmetic composition is for treating a skin condition.

According to another aspect of some embodiments of the present invention, there is provided a method of treating a skin condition in a subject in need thereof, which includes administering to the subject an effective amount of the molecular structure provided herein or the cosmetic composition provided herein.

In some embodiments, the skin condition treatable by the molecular structure provided herein, is selected from the group consisting of melasma, skin whitening, hyperpigmentation, Chadwick's sign, Linea alba, Perineal raphe, acne scarring, acne, liver spots, surgical scars, stretch marks and hair loss.

According to another aspect of some embodiments of the present invention, there is provided a process of preparing the molecular structure provided herein, which is effected by:
i) attaching D₁ to HA via L₁;
ii) attaching D₂ to D₁ via L₂; and
iii) for n greater than 2, optionally attaching Dₙ to D₂ or to Dₙ₋₁ via L₂ or Dₙ;
and/or
iv) attaching D₂ to D₁ via L₂;
v) for n greater than 2, optionally attaching Dₙ to D₂ or to Dₙ₋₁ via L₂ or Dₙ; and
vi) attaching D₁ to HA via L₁.

In some embodiments, the process further includes, prior to Step (i) and/or Step (vi), modifying at least one functional group in HA so as to exhibit at least one functional group modification moiety.

In some embodiments, the process further includes attaching a second hyaluronic acid moiety to the molecular structure via Lₙ.

In some embodiments of any one of the aspects presented herein, at least one of D₁, D₂ . . . and Dₙ is a precursor or a prodrug of a bioactive agent.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings and images in detail, it is stressed that the particulars shown are by way of example and for purposes of illustrative discussion of embodiments of the invention. In this regard, the description taken with the drawings and images makes apparent to those skilled in the art how embodiments of the invention may be practiced.

In the drawings:

FIGS. 1A-E present schematic illustrations of several exemplary non-limiting embodiments of the herein-provided molecular structure, wherein FIG. 1A presents an exemplary embodiment of a single HA moiety having multiple strings of bioactive agent moieties (denoted by circles) linked to the HA moiety and to one-another by biocleavable linking moieties (denoted by ovals), FIG. 1B presents a molecular structure comprising two HA moieties crosslinked by a string of bioactive agent moieties and further having non-crosslinking strings attached thereto, FIG. 1C presents a molecular structure comprising multiple HA moieties attached to some crosslinking strings, FIG. 1D presents a molecular structure comprising two HA moieties crosslinked by multiple crosslinking strings of bioactive agent moieties, and FIG. 1E presents a molecular structure comprising multiple HA moieties crosslinked by multiple crosslinking strings of bioactive agent moieties; and

FIG. 2 presents a schematic representation of steps in several processes for preparing the molecular structure provided herein (10), according to some embodiments of the present invention, wherein Step 11 shows an optional modification of a hyaluronic acid strand by a functional group modification moiety, Step 12 shows the attachment of the first bioactive agent moiety via a first biocleavable linking moiety to the HA moiety optionally having a modified functional group, Step 13 shows the attachment of a second bioactive agent moiety via a second biocleavable linking moiety to the first bioactive agent moiety, Step 14 shows the optional elongation of the string with additional bioactive agent moieties, Step 15 the optional crosslinking of at least two molecular structures via a biocleavable linking moiety to form a crosslinking string, and Steps 16 and Step 17 shows the optional attachment of a second hyaluronic acid moiety to some of the strings of a molecular structure thereby forming a crosslinked molecular structure.

### DESCRIPTION OF EMBODIMENTS OF THE INVENTION

The present invention, in some embodiments thereof, relates to cosmetics, and more particularly, but not exclusively, to a molecular structure based on hyaluronic acid that acts as a multiple-active agent delivery vehicle, and to uses thereof.

Before explaining at least one embodiment of the invention in detail, it is to be understood that the invention is not necessarily limited in its application to the details set forth in the following description or exemplified by the Examples. The disclosure is meant to encompass other embodiments or of being practiced or carried out in various ways.

While conceiving the present invention, the present inventors envisioned a molecular structure that consist of hyaluronic acid and several different bioactive agent residues, or moieties, linked in a linear chain via biocleavable linking moieties to the HA molecule. HA lends itself for many chemical modifications via its reactive functional groups and can be used as a single-strand polysaccharide, a double stranded polymeric conjugate, or a crosslinked complex comprising more than two strands.

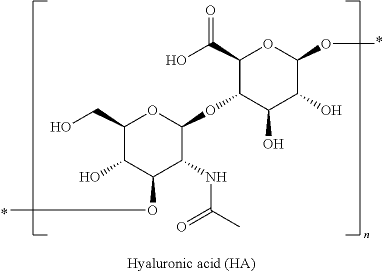

The present inventors have envisioned a molecular structure which, according to some embodiments, includes one or more HA strands, bearing a linear, a branched or a dendrimeric string of bioactive agents, linked to HA and to one another via biocleavable linkers, a.k.a. biodegradable moieties, thereby rendering HA a drug-delivery vehicle. The gist of the molecular structure is therefore a unique macromolecule that essentially has the physicomechanical and biochemical properties of HA, having one or more stings of moieties of different bioactive agents linked by biocleavable linkers, which can be used to target the bodily site, such as the skin, for delivery of various bioactive agents attached to the HA, whereas upon biocleavage, releases the bioactive agents.

### Molecular Structure

Thus, according to some embodiments of the present invention, there is provided a molecular structure, comprising one of more hyaluronic acid strands and one or more linear and/or branched/dendrimeric strings of releasable bioactive agents attached therebetween and to the HA strand(s) via biocleavable linking moieties.

In some embodiments, the molecular structure can be represented by general Formula I:

HA [symbol] L₁-D₁-L₂-D₂ . . . Lₙ-Dₙ]ₘ Formula I
wherein:
HA is a hyaluronic acid moiety, referred to herein as the first HA moiety;
L₁, L₂ . . . and Lₙ are each independently a biocleavable linking moiety;
D₁, D₂ . . . and Dₙ are each independently an bioactive agent moiety;
n is an integer equal or larger than 2; and
m is a positive number equal or larger than 1.

As used herein, the terms “moiety” and “residue”, used interchangeably, describe a portion of a molecule, and typically a major portion thereof, or a group of atoms pertaining to a specific function. In general, the molecular structure provided herein comprises three structural elements, one or more hyaluronic acid strands, two or more bioactive agents, and biocleavable linking moiety that link the first two elements—the terms “moiety” and “residue” are used to refer to the first two elements in their bound form, and to the group of atoms that links these elements. In the context of the present invention, there terms are used to refer, for example, to a bioactive agent in its covalently bound form, as part of a molecular structure; in this example, a bioactive agent is released from the molecular structure when the biocleavable linking moiety that links the bioactive agent moiety to the molecular structure is cleaved.

The variable of “n” may also range higher than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50. In some embodiments of the present invention, n=2, 3, 4, 5, 6, 7, 8 or 9. In some embodiments, n=2-5.

The variable of “m” represents the number of strings of bioactive agent moieties that are attached to a HA moiety in the molecular structure provided herein. As discussed hereinbelow, since the process of forming the molecular structure has a statistic nature, the number of linear and/or branched/dendrimeric strings that are attached to a HA moiety cannot be determined deterministically, but rather assessed by analytical methods to be given range values or percentage of load per an average size of the HA moiety. As such, it is understood that in practice the value of “m” of formula I can also be expressed in terms of a range, an average, or as percentage relative to the HA moiety.

The rationale behind this conceptual molecular structure is based on the following:
i) HA moieties can be linked to various small molecular moieties via several functional groups having varied reactivity, thereby HA lends itself to chemical modifications which can be tailor made for specific applications;
ii) HA can be used as a carrier to deliver bioactive agents that are relevant for cosmetic and pharmaceutical treatments in relevant bodily sites (e.g., skin);
iii) Bioactive agents exhibit functional groups which are conducive to conjugation via biocleavable linking moieties;
iv) Biocleavable linking moieties can be selected to suit a site-specific or enzyme-specific release design, such that bioactive agents linked to HA and to one-another via such enzymatically cleavable linking moieties can be cleaved enzymatically in vivo at a pre-selected targeted tissue/organ/bodily site;
v) The release profile of each of the bioactive agents depends, inter alia, on the nature of the nature of the bioactive agent, the location of the bioactive agent in the string, the biocleavable linking moiety, the macromolecular structure of the molecular structure (e.g., single stranded HA, double stranded HA, or randomly crosslinked HA), and the physiological environment (enzymes and other factors);
vi) In some embodiments, all the breakdown products of the molecular structure are useful for the intended use per-se.

Since the attachment of several moieties on a plurality of functional groups along a large polymeric entity like HA is not a deterministic process but a statistical process, the molecular structures can be characterized also by the percentage of elements present in the structure. An assessment of the percentage of elements present in the structure can be effected, for example, by submitting a batch of the molecular structure, according to some embodiments of the present invention, to total degradation by hyaluronidase in D₂O and determination of the percentage of the string-load by proton NMR comparing the area under the relevant peaks. A similar approach can be effected for determination of the percentage of bioactive agent load on the molecular structure, by submitting the same to total cleavage of all linking moieties, and following detectable markers for each bioactive agent released from the molecular structure. For example, 10% loading of a certain bioactive agent means area under the peak associated with HA is 10 times greater than the area under the peak associated with the bioactive agent.

In some embodiments, the molecular structure comprises a single strand of HA (a first HA moiety) and at least one linear and/or branched/dendrimeric string of bioactive agent moieties, represented in Formula I as L₁-D₁-L₂-D₂ . . . and Lₙ-Dₙ, wherein Dₙ is the last bioactive agent moiety in the string.

In some embodiments, the molecular structure comprises two or more HA moieties, being crosslinked with one or more strings of releasable bioactive agents. As can be seen in Formula II, Lₙ₊₁ is a linking moiety that links the string on a first HA moiety HA₁ to a second hyaluronic acid moiety HA₂, thereby forming a crosslinking string of releasable bioactive agent moieties. Without being bound by any particular theory, it is assumed that these crosslinked HA-based structures will be more stable and last longer under physiologic and metabolic conditions, and thus allow controlled and sustained release of the releasable moieties in the structure.

In some embodiments of the present invention, the molecular structure that comprises crosslinked HA moieties can be represented by general Formula II:

HA₁[-L₁-D₁-L₂-D₂ . . . Lₙ-Dₙ-Lₙ₊₁-HA₂]ₘ Formula II
wherein:
HA₁ is said first hyaluronic acid moiety; and
L₁, L₂ . . . and Lₙ are each independently a biocleavable linking moiety;
D₁, D₂ . . . and Dₙ are each independently an bioactive agent moiety;
Lₙ₊₁ is a biocleavable linking moiety or absent in some of the strings but not absent in all the strings;
HA₂ is said second hyaluronic acid moiety or absent in some of the strings but not absent in all the strings;
n is an integer equal or larger than 2; and
m is a positive number equal or larger than 1, defined as discussed hereinabove.

In some embodiments, the molecular structure comprises at least two HA moieties being crosslinked with one or more strings of releasable bioactive agents, wherein the molecular structure is formed by forming a biocleavable moiety between two bioactive agent moieties at the end of a string which has been attached to the HA moieties prior to the crosslinking reaction. In such embodiments, a linking moiety is formed by conjugating the last bioactive agent moieties situated at end of a string on HA moieties of two molecular structures, thereby forming a crosslinking string of releasable bioactive agent moieties.

In some embodiments, the molecular structure comprises more than two HA moieties being crosslinked to one another via the ends of strings on each HA moiety, either directly or by conjugating two strings into a crosslinking string of releasable bioactive agent moieties.

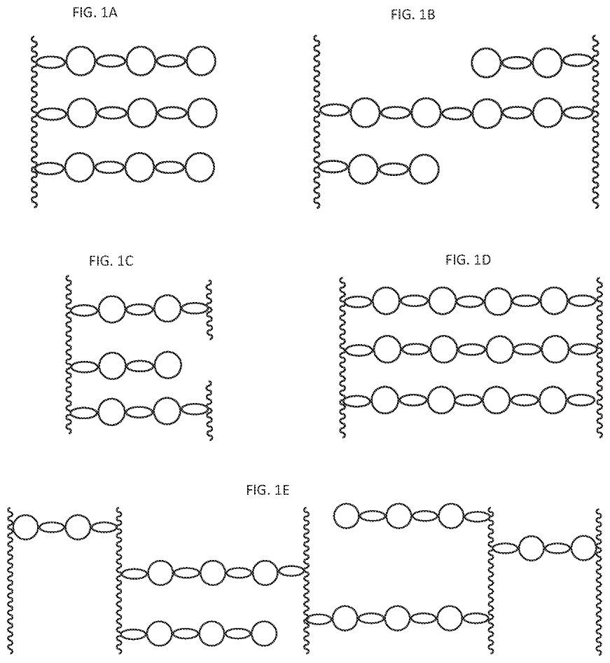

FIGS. 1A-E present schematic illustrations of several exemplary non-limiting embodiments of the herein-provided molecular structure, wherein FIG. 1A presents an exemplary embodiment of a single HA moiety having multiple strings of bioactive agent moieties (denoted by circles) linked to the HA moiety and to one-another by biocleavable linking moieties (denoted by ovals), FIG. 1B presents a molecular structure comprising two HA moieties crosslinked by a string of bioactive agent moieties and further having non-crosslinking strings attached thereto, FIG. 1C presents a molecular structure comprising multiple HA moieties attached to some crosslinking strings, FIG. 1D presents a molecular structure comprising two HA moieties crosslinked by multiple crosslinking strings of bioactive agent moieties, and FIG. 1E presents a molecular structure comprising multiple HA moieties crosslinked by multiple crosslinking strings of bioactive agent moieties.

According to some embodiments, the molecular structure includes one or more HA moieties and one or more strings of releasable bioactive agents, whereas the HA moieties and the string(s) contain no other releasable element which is not hyaluronic acid or a bioactive agent. In other words, the molecular structure comprises only moieties of HA and bioactive agents, whereupon biocleavage, release essentially only HA and bioactive agents.

For any one of the embodiments described herein, the molecular structures described herein may be in a form of a salt, for example, a cosmetically and/or pharmaceutically acceptable salt. As used herein, the phrase “cosmetically and/or pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound.

In the context of some of the present embodiments, a cosmetically and/or pharmaceutically acceptable salt of the compounds described herein may optionally be a base addition salt comprising at least one acidic (e.g., carboxylic acid) group of the compound which is in a negatively charged form, e.g., wherein the acidic group is deprotonated, in combination with at least one counter-ion, derived from the selected base, that forms a pharmaceutically acceptable salt.

The base addition salts of the compounds described herein may therefore be complexes formed between one or more acidic groups of the drug and one or more equivalents of a base.

The base addition salts may include a variety of organic and inorganic counter-ions and bases, such as, but not limited to, sodium (e.g., by addition of NaOH), potassium (e.g., by addition of KOH), calcium (e.g., by addition of Ca(OH)₂, magnesium (e.g., by addition of Mg(OH)₂), aluminum (e.g., by addition of Al(OH)₃ and ammonium (e.g., by addition of ammonia). Each of these acid addition salts can be either a mono-addition salt or a poly-addition salt, as these terms are defined herein.

Depending on the stoichiometric proportions between the charged group(s) in the compound and the counter-ion in the salt, the acid or base additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

Further, each of the compounds described herein, including the salts thereof, can be in a form of a solvate or a hydrate thereof.

The compounds described herein can be used as polymorphs and the present embodiments further encompass any isomorph of the compounds and any combination thereof.

The present embodiments further encompass any enantiomers, prodrugs, solvates, hydrates and/or pharmaceutically acceptable salts of the molecular structures described herein and methods, compositions and uses utilizing enantiomers, diastereomers, prodrugs, solvates, hydrates and/or pharmaceutically acceptable salts of the molecular structures described herein.

The term “prodrug” refers to an agent, which is converted into a bioactive agent (the active parent drug) in vivo. In essence, the entire molecular structures presented herein constitute a form of a prodrug, as drug moieties, which are designed for release as bioactive agents in a controllable manner, are linked thereto. Prodrugs are typically useful for facilitating and/or targeting the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of a bioactive agent in vivo. An example, without limitation, of a prodrug would be a bioactive agent, according to some embodiments of the present invention, having one or more carboxylic acid moieties, which is administered as an ester (the “prodrug”). Such a prodrug is hydrolyzed in vivo, to thereby provide the free bioactive agent (the parent drug). The selected ester may affect both the solubility characteristics and the hydrolysis rate of the prodrug. A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C1-4)acyl (e.g., acetyl) group to form an ester group, and/or any one or more of the carboxylic acid groups of the compound is modified by an alkoxy or aryloxy group, optionally (C1-4)alkoxy (e.g., methyl, ethyl) group to form an ester group.

The term “solvate” refers to a complex of variable stoichiometric (e.g., di-, tri-, tetra-, penta-, hexa-, and so on), which is formed by a solute (the molecular structures described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

### Bioactive Agent

As discussed hereinabove, the molecular structures is designed to carry a multiple-drug payload, which can comprise several copies of the same drug, linked by similar or different linking moieties, to control the release profile of the payload, or comprise of a series of different drugs linked by similar or different linking moieties. In some embodiments where the drugs are the same, the molecular structures of the present invention provide for substantial enhancement of the functionality of the bioactive agent, both in terms of localized release, concerted release or prolonged sequential release thereof. In some embodiments where the bioactive agents are different from one-another, the molecular structures of the present invention provides for simultaneous, concerted or sequential release of the drugs and can therefore be specifically advantageous in cases where the different bioactive agents confer a cumulative and/or a synergistic effect.

In some embodiments, each of the bioactive agents in the string attached to HA is the same bioactive agent, namely each of D₁, D₂ . . . and Dₙ in Formula I is a moiety of the same bioactive agent.

In some embodiments, each of the bioactive agents in the string attached to HA is a different bioactive agent, namely each of D₁, D₂ . . . and Dₙ in Formula I is a moiety of a different (non-similar) bioactive agent.

In the context of the present embodiments, the terms “bioactive agent”, “cosmetically active agent”, “pharmaceutically active agent”, “active agent” and “drug” are used interchangeably.

As used herein, the terms “bioactive agent” and “drug” refer to small molecules or biomolecules that alter, inhibit, activate, or otherwise affect a biological mechanism or event. Bioactive agent that can be tethered as a linear and/or branched/dendrimeric string to HA, according to embodiments of the present invention, include, but are not limited to various compounds that confer a beneficial effect on various skin conditions, such as, without limitation, melasma.

As used herein, the term “small molecule” refers to molecules, whether naturally-occurring or artificially created (e.g., via chemical synthesis), that have a relatively low molecular weight. Typically, small molecules are monomeric and have a molecular weight of less than about 1500 Da. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, are all considered acceptable for use in accordance with the present invention.

Exemplary, non-limiting examples of bioactive agents that can be releasably linked to HA, include tranexamic acid, kojic acid, cysteamine, cystamine, azelaic acid, hydroquinone, mequinol, flutamide, CBD, arbutin, vitamin A, vitamin C, vitamin E, ellagic acid, glutathione, biotin, mandelic acid, an alpha-hydroxy acid, tretinoin, an alpha-lipoic acid, glutathione, salicylic acid and fluocinolone.

A more comprehensive listing of exemplary bioactive agents that may be suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmcopeial Convention, Inc., Rockville Md., 2001.

Anti-inflammatory drugs that can be linked and controllably released from the molecular structure according to some embodiments of the invention include, but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

Suitable antimicrobial agents, including antibacterial, antifungal, antiprotozoal and antiviral agents, for use in context of the present invention include, without limitation, beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, farnesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, parachlorometa xylenol, nystatin, tolnaftate and clotrimazole and mixtures thereof.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the trade name Trolox^{R}), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B₃ (niacinamide and its derivatives), alpha hydroxy acids (such as glycolic acid, lactic acid, tartaric acid, malic acid, citric acid, etc.) and beta hydroxy acids (such as salicylic acid and the like).

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

Representative examples of hormones include, without limitation, methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5α-dihydrotestosterone, testolactone, 17α-methyl-19-nortestosterone and pharmaceutically acceptable esters and salts thereof, and combinations of any one of the foregoing.

Non-limiting examples of analgesic agents that can be efficiently delivered by the molecular structures of the present invention, include acetaminophen, alfentanil hydrochloride, aminobenzoate potassium, aminobenzoate sodium, anidoxime, anileridine, anileridine hydrochloride, anilopam hydrochloride, anirolac, antipyrine, aspirin, benoxaprofen, benzydamine hydrochloride, bicifadine hydrochloride, brifentanil hydrochloride, bromadoline maleate, bromfenac sodium, buprenorphine hydrochloride, butacetin, butixirate, butorphanol, butorphanol tartrate, carbamazepine, carbaspirin calcium, carbiphene hydrochloride, carfentanil citrate, ciprefadol succinate, ciramadol, ciramadol hydrochloride, clonixeril, clonixin, codeine, codeine phosphate, codeine sulfate, conorphone hydrochloride, cyclazocine, dexoxadrol hydrochloride, dexpemedolac, dezocine, diflunisal, dihydrocodeine bitartrate, dimefadane, dipyrone, doxpicomine hydrochloride, drinidene, enadoline hydrochloride, epirizole, ergotamine tartrate, ethoxazene hydrochloride, etofenamate, eugenol, fenoprofen, fenoprofen calcium, fentanyl citrate, floctafenine, flufenisal, flunixin, flunixin meglumine, flupirtine maleate, fluproquazone, fluradoline hydrochloride, flurbiprofen, hydromorphone hydrochloride, ibufenac, indoprofen, ketazocine, ketorfanol, ketorolac tromethamine, letimide hydrochloride, levomethadyl acetate, levomethadyl acetate hydrochloride, levonantradol hydrochloride, levorphanol tartrate, lofemizole hydrochloride, lofentanil oxalate, lorcinadol, lornoxicam, magnesium salicylate, mefenamic acid, menabitan hydrochloride, meperidine hydrochloride, meptazinol hydrochloride, methadone hydrochloride, methadyl acetate, methopholine, methotrimeprazine, metkephamid acetate, mimbane hydrochloride, mirfentanil hydrochloride, molinazone, morphine sulfate, moxazocine, nabitan hydrochloride, nalbuphine hydrochloride, nalmexone hydrochloride, namoxyrate, nantradol hydrochloride, naproxen, naproxen sodium, naproxol, nefopam hydrochloride, nexeridine hydrochloride, noracymethadol hydrochloride, ocfentanil hydrochloride, octazamide, olvanil, oxetorone fumarate, oxycodone, oxycodone hydrochloride, oxycodone terephthalate, oxymorphone hydrochloride, pemedolac, pentamorphone, pentazocine, pentazocine hydrochloride, pentazocine lactate, phenazopyridine hydrochloride, phenyramidol hydrochloride, picenadol hydrochloride, pinadoline, pirfenidone, piroxicam olamine, pravadoline maleate, prodilidine hydrochloride, profadol hydrochloride, propiram fumarate, propoxyphene hydrochloride, propoxyphene napsylate, proxazole, proxazole citrate, proxorphan tartrate, pyrroliphene hydrochloride, remifentanil hydrochloride, salcolex, salethamide maleate, salicylamide, salicylate meglumine, salsalate, sodium salicylate, spiradoline mesylate, sufentanil, sufentanil citrate, talmetacin, talniflumate, talosalate, tazadolene succinate, tebufelone, tetrydamine, tifurac sodium, tilidine hydrochloride, tiopinac, tonazocine mesylate, tramadol hydrochloride, trefentanil hydrochloride, trolamine, veradoline hydrochloride, verilopam hydrochloride, volazocine, xorphanol mesylate, xylazine hydrochloride, zenazocine mesylate, zomepirac sodium and zucapsaicin.

Anti-cancer drugs that can be linked and controllably released from the molecular structure according to some embodiments of the invention include, but are not limited to Chlorambucil; 3-(9-Acridinylamino)-5-(hydroxymethyl)aniline; Azatoxin; Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Non-limiting examples of chemotherapeutic agents that can be efficiently delivered by the molecular structures of the present invention, include amino containing chemotherapeutic agents such as camptothecin, daunorubicin, doxorubicin, N-(5,5-diacetoxypentyl)doxorubicin, anthracycline, mitomycin C, mitomycin A, 9-amino aminopertin, antinomycin, N⁸-acetyl spermidine, 1-(2-chloroethyl)-1,2-dimethanesulfonyl hydrazine, bleomycin, tallysomucin, and derivatives thereof; hydroxy containing chemotherapeutic agents such as etoposide, irinotecan, topotecan, 9-amino camptothecin, paclitaxel, docetaxel, esperamycin, 1,8-dihydroxy-bicyclo[7.3.1]trideca-4-ene-2,6-diyne-13-one, anguidine, morpholino-doxorubicin, vincristine and vinblastine, and derivatives thereof, sulfhydril containing chemotherapeutic agents and carboxyl containing chemotherapeutic agents. Additional chemotherapeutic agents include, without limitation, an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, a gonadotropin-releasing hormone analog, bleomycin, doxorubicin, paclitaxel, 4-OH cyclophosphamide and cisplatinum.

The molecular structure provided herein may carry, deliver and release bioactive agents in the form of prodrugs, in order to protect the bioactive agent from degradation prior to release from the molecular structure prematurely. A prodrug is a medication or compound that, after administration, is metabolized (i.e., converted within the body) into a pharmacologically and/or biologically active agent. Instead of administering a drug directly, a corresponding prodrug can be used to improve how the drug is absorbed, distributed, metabolized, and excreted (ADME). Hence, according to some embodiments of the present invention, the term “bioactive agent” encompasses a prodrug or a precursor (forerunner) of a bioactive agent. In such embodiments, the bioactive agent is seen as having “protecting groups” on one or more of its functional groups, which are removed metabolically once the bioactive agent precursor is releases from the molecular structure, thereby affording a released bioactive agent. For example of a prodrug of a bioactive agent which exhibits a carboxylic group that may prematurely degrade, whereas the prodrug exhibits an ester in the same position, which will metabolize to a carboxylic group once the prodrug is released. Additional information and guidance regarding prodrugs and protecting groups in this regard, can be found, for example, in “A New Classification of Prodrugs: Regulatory Perspectives” [Wu, K. M. Pharmaceuticals (Basel), 2009, 2(3), pp. 77-81].

In some embodiments, the term “bioactive agent” encompasses a bioactive agent precursor that can undergo a metabolic chemical transformation to afford a bioactive agent; in other words, the precursor is a bioactive agent that can further undergo a biochemical reaction in the physiological environment to afford a derivative bioactive agent.

### Biocleavable Inking Moiety

As used herein, the term “linking moiety” describes a chemical moiety (a group of atoms or a covalent bond) that links two chemical moieties via one or more covalent bonds. A linking moiety may include atoms that form a part of one or both of the chemical moieties it links, and/or include atoms that do not form a part of one or both of the chemical moieties it links. For example, a peptide bond (amide) linking moiety that links two bioactive agent moieties includes at least a nitrogen atom and a hydrogen atom from one bioactive agent moiety and at least a carboxyl of the other bioactive agent moiety. In general, the linking moiety can be formed during a chemical reaction, such that by reacting two or more reactive groups, the linking moiety is formed as a new chemical entity which can comprise a bond (between two atoms), or one or more bonded atoms. Alternatively, the linking moiety can be an independent chemical moiety comprising two or more reactive groups to which the reactive groups of other compounds can be attached, either directly or indirectly, as is detailed hereinunder.

The positions at which the bioactive agent is linked to the molecular structure presented herein are generally selected such that once cleaved off the molecular structure, any remaining moiety stemming from the linking moiety on the bioactive agent, if at all, does not substantially preclude its biological activity (mechanism of biological activity). Suitable positions depend on the type of bioactive agent. According to some embodiments of the present invention, the linking moieties are form such that the biological activity of the bioactive agent, once released from the molecular structure, is not abolished and remains substantially the same as the biological activity of a similar pristine bioactive agent. According to some embodiments of the present invention, the linking moiety is such that once a bioactive agent is released from the molecular structure, it is a pristine bioactive agent.

In some embodiments, the term “linking moiety” is defined so as not to encompass a moiety that once the linking moiety is cleaved, standalone molecule is released. This limitation excludes linking moiety that releases upon cleavage standalone molecules such water molecules, gas molecules, small organic ions, such as acetate, small inorganic ions such as hydroxide, and the likes. In such embodiments, the molecular structure may be regarded as one that does not release non-bioactive agents.

As used herein, the words “link”, “linked”, “linkage” “linker”, “bound” or “attached”, are used interchangeably herein and refer to the presence of at least one covalent bond between species, unless specifically noted otherwise.

In some embodiments, each of the linking moieties in the string or a crosslinking string is the same biocleavable linking moiety.

In some embodiments, each of the linking moieties in the string or a crosslinking string is a different biocleavable linking moiety. In some of these embodiments, each of of the linking moieties in the string or a crosslinking string is also characterized by a different biocleavage condition.

The term “biocleavage” refers to a biochemical reaction that causes the linking moiety to break/dissociate. In the context of embodiments of the present invention, biocleavage is typically mediated by biomolecules (e.g., enzymes, RNA and the likes) in an organism or an organ thereof, whereas each such mediator is active or more active under certain conditions, including location in the organism (cell, tissue, organ), temperature, pH, ionic strength, light and other reaction effectors, as these are known in the art. Linking moieties having different biocleavage condition allow the release of bioactive agents at different locations in the subject, and/or different times, and/or under otherwise different physiological conditions.

As stated hereinabove, a linking moiety can be formed during a chemical reaction, such that by reacting two or more reactive groups. The phrase “reactive group”, as used herein, refers to a chemical group that is capable of undergoing a chemical reaction that typically leads to the formation a covalent bond. Chemical reactions that lead to a bond formation include, for example, cycloaddition reactions (such as the Diels-Alder's reaction, the 1,3-dipolar cycloaddition Huisgen reaction, and the similar “click reaction”), condensations, nucleophilic and electrophilic addition reactions, nucleophilic and electrophilic substitutions, addition and elimination reactions, alkylation reactions, rearrangement reactions and any other known organic reactions that involve a reactive group.

Representative examples of reactive groups include, without limitation, acyl halide, aldehyde, alkoxy, alkyne, amide, amine, aryloxy, azide, aziridine, azo, carbamate, carbonyl, carboxyl, carboxylate, cyano, diene, dienophile, epoxy, guanidine, guanyl, halide, hydrazide, hydrazine, hydroxy, hydroxylamine, imino, isocyanate, nitro, phosphate, phosphonate, sulfinyl, sulfonamide, sulfonate, thioalkoxy, thioaryloxy, thiocarbamate, thiocarbonyl, thiohydroxy, thiourea and urea, as these terms are defined hereinafter.

Linking moieties, according to some embodiments of the present invention, include without limitation, —O—, —S—, —S—S—, —Se—, —Se—Se—, —NH—NH—, —NH—, —NH—(CH₂)₂—NH—, —O—CH₂—O—, —O—(CH₂)₂—O—, —(C═O)—NH—, —(C═O)—O—, —(C═O)—S—, —C(═NH)—NH—, —(O═O)—NH—, —P(OH)(═O)—NH— and —NH—NH(C═O)—NH—.

Additional non-limiting examples of linking moieties, according to some embodiments of the present invention, include, amide, carbamate, carbonate, lactone, lactam, carboxylate, ester, cycloalkene, cyclohexene, heteroalicyclic, heteroaryl, triazine, triazole, disulfide, imine, imide, oxime, aldimine, ketimine, hydrazone, semicarbazone, acetal, ketal, aminal, aminoacetal, thioacetal, thioketal, phosphate ester, and the like. Other linking moieties are defined hereinbelow, and further other linking moieties are contemplated within the scope of the term as used herein.

According to some embodiments, the linking moiety is selected from the group consisting of:

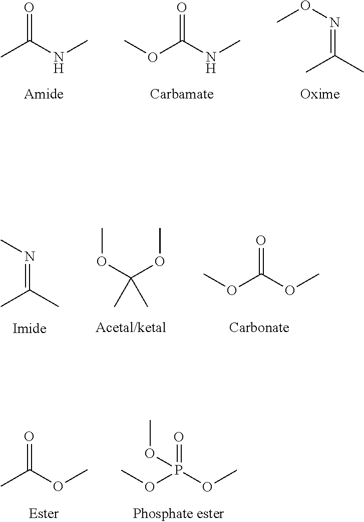

In some embodiments, the HA stand of the molecular structure provided herein, undergoes modification of one of more native functional groups on the HA strand in order to increase the efficiency of attachment bulky bioactive agent(s) or linear and/or branched/dendrimeric strings thereof, or introduce a functionality to the HA strand that can react with compatible function group(s) in the bioactive agent(s) or strings thereof. When forming a part of the molecular structure, such linking moieties are afforded by reacting a functional group modificator with HA, and the resulting linking moiety is referred to herein as a functional group modification moiety. Exemplary functional group modificators that result in the introduction of an amide functionality to the HA moiety include, without limitation, Gly, β-Ala, GABA, 3-Amino-2,2-dimethyl-propionic acid, sarcosine, and NH₂-PEG4-Propionic acid. Exemplary functional group modificators that result in the modification of a carboxyl in the HA moiety into an ester functionality include, without limitation, hydroxyacetic acid, hydroxypropanoic acid, and hydroxybenzoic acid. Exemplary functional group modificators that result in the modification of a hydroxyl in the HA moiety into an ester functionality include, without limitation, succinic acid, glutaric acid, adipic acid, and phthalic acid. Exemplary functional group modificators that result in the modification of a carboxyl in the HA moiety into a hydrazide functionality include, without limitation, glycine hydrazide, alanine hydrazide, and β-alanine hydrazide.

Definitions of specific functional groups, chemical terms, and general terms used throughout the specification are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987.

As used herein, the terms “amine” or “amino”, describe both a —NR′R″ end group and a —NR′— linking moiety, wherein R′ and R″ are each independently hydrogen, alkyl, cycloalkyl, aryl, as these terms are defined hereinbelow.

Herein throughout, the phrase “end group” describes a chemical group that is attached to one compound (a substituent; a reactive group; a functional group etc.), while the term “linking moiety” describes a group that is attached to two compounds and links therebetween.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydrogen, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine, as these terms are defined herein.

The term “alkyl” describes a saturated aliphatic hydrocarbon including straight chain (unbranched) and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking moiety, as this phrase is defined hereinabove, which connects two or more moieties via at least two carbons in its chain. When an alkyl is a linking moiety, it is also referred to herein as “alkylene”, e.g., methylene, ethylene, propylene, etc.

The term “alkenyl” describes an unsaturated alkyl, as defined herein, having at least two carbon atoms and at least one carbon-carbon double bond. The alkenyl may be substituted or unsubstituted by one or more substituents, as described for alkyl hereinabove.

The terms “alkynyl” or “alkyne”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings that share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking moiety, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking moiety, as this phrase is defined hereinabove, connecting two or more moieties at two or more positions thereof. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking moiety, as this term is defined hereinabove, connecting two or more moieties at two or more positions thereof. Preferably, the aryl is phenyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halo, sulfonate, sulfoxide, phosphonate, hydroxy, alkoxy, aryloxy, thiohydroxy, thioalkoxy, thioaryloxy, cyano, nitro, azo, azido, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking moiety, as this phrase is defined hereinabove, connecting two or more moieties at two or more positions thereof. Representative examples are pyridine, pyrrole, oxazole, indole, purine and the like.

The term “alkaryl” describes an alkyl, as defined herein, which is substituted by one or more aryl or heteroaryl groups. An example of alkaryl is benzyl.

The term “amine-oxide” describes a —N(OR′)(R″) or a —N(OR′)— group, where R′ and R″ are as defined herein. This term refers to a —N(OR′)(R″) group in cases where the amine-oxide is an end group, as this phrase is defined hereinabove, and to a —N(OR′)— group in cases where the amine-oxime is an end group, as this phrase is defined hereinabove.

As used herein, the term “acyl” refers to a group having the general formula —C(═O)R′, —C(═O)OR′, —C(═O)—O—C(═O)R′, —C(═O)SR′, —C(═O)N(R′)₂, —C(═S)R′, —C(═S)N(R′)₂, and —C(═S)S(R′), —C(═NR′)R″, —C(═NR′)OR″, —C(═NR′)SR″, and —C(═NR′)N(R″)₂, wherein R′ and R″ are each independently hydrogen, halo, substituted or unsubstituted hydroxyl, substituted or unsubstituted thiol, substituted or unsubstituted amine, substituted or unsubstituted acyl, cyclic or acyclic, substituted or unsubstituted, branched or unbranched aliphatic, cyclic or acyclic, substituted or unsubstituted, branched or unbranched heteroaliphatic, cyclic or acyclic, substituted or unsubstituted, branched or unbranched alkyl, cyclic or acyclic, substituted or unsubstituted, branched or unbranched alkenyl, substituted or unsubstituted alkynyl, substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any one of the substituents described herein, that result in the formation of a stable moiety (e.g., aliphatic, alkyl, alkenyl, alkynyl, heteroaliphatic, heterocyclic, aryl, heteroaryl, acyl, oxo, imino, thioxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “aliphatic” or “aliphatic group” denotes an optionally substituted hydrocarbon moiety that may be straight-chain (i.e., unbranched), branched, or cyclic (“carbocyclic”) and may be completely saturated or may contain one or more units of unsaturation, but which is not aromatic. Unless otherwise specified, aliphatic groups contain 1-12 carbon atoms. In some embodiments, aliphatic groups contain 1-6 carbon atoms. In some embodiments, aliphatic groups contain 1-4 carbon atoms, and in yet other embodiments aliphatic groups contain 1-3 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

As used herein, the terms “heteroaliphatic” or “heteroaliphatic group”, denote an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. Unless otherwise specified, heteroaliphatic groups contain 1-6 carbon atoms wherein 1-3 carbon atoms are optionally and independently replaced with heteroatoms selected from oxygen, nitrogen and sulfur. In some embodiments, heteroaliphatic groups contain 1-4 carbon atoms, wherein 1-2 carbon atoms are optionally and independently replaced with heteroatoms selected from oxygen, nitrogen and sulfur. In yet other embodiments, heteroaliphatic groups contain 1-3 carbon atoms, wherein 1 carbon atom is optionally and independently replaced with a heteroatom selected from oxygen, nitrogen and sulfur. Suitable heteroaliphatic groups include, but are not limited to, linear or branched, heteroalkyl, heteroalkenyl, and heteroalkynyl groups.

The term “halo” describes fluorine, chlorine, bromine or iodine substituent.

The term “halide” describes an anion of a halogen atom, namely F⁻, Cl⁻ Br⁻ and I⁻.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking moiety, as these phrases are defined hereinabove, where R′ is as defined hereinabove.

The term “thiosulfate” describes a —O—S(═S)(═O)—OR′ end group or a —O—S(═S)(═O)—O— linking moiety, as these phrases are defined hereinabove, where R′ is as defined hereinabove.

The term “sulfite” describes an —O—S(═O)—O—R′ end group or a —O—S(═O)—O— group linking moiety, as these phrases are defined hereinabove, where R′ is as defined hereinabove.

The term “thiosulfite” describes a —O—S(═S)—O—R′ end group or an —O—S(═S)—O— group linking moiety, as these phrases are defined hereinabove, where R′ is as defined hereinabove.

The term “sulfinate” or “sulfinyl” describes a —S(═O)—OR′ end group or an —S(═O)—O— group linking moiety, as these phrases are defined hereinabove, where R′ is as defined hereinabove.

The terms “solfoxide” or “sulfinyl” describe a —S(═O)R′ end group or an —S(═O)— linking moiety, as these phrases are defined hereinabove, where R′ is as defined hereinabove.

The term “sulfonate” or “sulfonyl” describes a —S(═O)₂—R′ end group or an —S(═O)₂— linking moiety, as these phrases are defined hereinabove, where R′ is as defined herein.

The term “S-sulfonamide” describes a —S(═O)₂—NR′R″ end group or a —S(═O)₂—NR′— linking moiety, as these phrases are defined hereinabove, with R′ and R″ as defined herein.

The term “N-sulfonamide” describes an R′S(═O)₂—NR″— end group or a —S(═O)₂—NR′— linking moiety, as these phrases are defined hereinabove, where R′ and R″ are as defined herein.

The term “disulfide” refers to a —S—SR′ end group or a —S—S— linking moiety, as these phrases are defined hereinabove, where R′ is as defined herein.

The term “phosphate” describes an —O—P(═O)₂(OR′) end or reactive group or a —O—P(═O)₂(O)— linking moiety, as these phrases are defined hereinabove, with R′ as defined herein.

The term “phosphonate” describes a —P(═O)(OR′) (OR″) end or reactive group or a —P(═O)(OR′)(O)— linking moiety, as these phrases are defined hereinabove, with R′ and R″ as defined herein.

The term “thiophosphonate” describes a —P(═S)(OR′) (OR″) end group or a —P(═S)(OR′)(O)— linking moiety, as these phrases are defined hereinabove, with R′ and R″ as defined herein.

The term “carbonyl” or “carbonate” as used herein, describes a —C(═O)—R′ end group or a —C(═O)— linking moiety, as these phrases are defined hereinabove, with R′ as defined herein.

The term “thiocarbonyl” as used herein, describes a —C(═S)—R′ end group or a —C(═S)— linking moiety, as these phrases are defined hereinabove, with R′ as defined herein.

The term “oxo” as used herein, described a ═O end group.

The term “thioxo” as used herein, described a ═S end group.

The term “oxime” describes a ═N—OH end group or a ═N—O— linking moiety, as these phrases are defined hereinabove.

The term “hydroxyl” describes a —OH group.

As used herein, the term “aldehyde” refers to an —C(═O)—H group.

The term “acyl halide” describes a —(C═O)R″″ group wherein R″″ is halo, as defined hereinabove.

The term “alkoxy” as used herein describes an —O-alkyl, an —O-cycloalkyl, as defined hereinabove. The ether group —O— is also a possible linking moiety.

The term “aryloxy” describes both an —O-aryl and an —O-heteroaryl group, as defined herein.

The term “disulfide” as used herein describes an —S—S— linking moiety, which in some cases forms between two thiohydroxyl groups.

The terms “thio”, “sulfhydryl” or “thiohydroxyl” as used herein describe an —SH group.

The term “thioalkoxy” or “thioether” describes both a —S-alkyl group, and a —S-cycloalkyl group, as defined herein. The thioether group —S— is also a possible linking moiety.

The term “thioaryloxy” describes both a —S-aryl and a —S-heteroaryl group, as defined herein. The thioarylether group —S-aryl- is also a possible linking moiety.

The term “cyano” or “nitrile” describes a —C≡N group.

The term “isocyanate” describes an —N═C═O group.

The term “nitro” describes an —NO₂ group.

The term “carboxylate” or “ester”, as used herein encompasses C-carboxylate and O-carboxylate.

The term “C-carboxylate” describes a —C(═O)—OR′ end group or a —C(═O)—O— linking moiety, as these phrases are defined hereinabove, where R′ is as defined herein.

The term “O-carboxylate” describes a —OC(═O)R′ end group or a —OC(═O)— linking moiety, as these phrases are defined hereinabove, where R′ is as defined herein.

The term “thiocarboxylate” as used herein encompasses “C-thiocarboxylate and O-thiocarboxylate.

The term “C-thiocarboxylate” describes a —C(═S)—OR′ end group or a —C(═S)—O— linking moiety, as these phrases are defined hereinabove, where R′ is as defined herein.

The term “O-thiocarboxylate” describes a —OC(═S)R′ end group or a —OC(═S)— linking moiety, as these phrases are defined hereinabove, where R′ is as defined herein.

The term “carbamate” as used herein encompasses N-carbamate and O-carbamate.

The term “N-carbamate” describes an R″OC(═O)—NR′— end group or a —OC(═O)—NR′— linking moiety, as these phrases are defined hereinabove, with R′ and R″ as defined herein.

The term “O-carbamate” describes an —OC(═O)—NR′R″ end group or an —OC(═O)—NR′— linking moiety, as these phrases are defined hereinabove, with R′ and R″ as defined herein.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking moiety, as these phrases are defined hereinabove, with R′ and R″ as defined herein.

The term “N-thiocarbamate” describes an R″OC(═S)NR′— end group or a —OC(═S)NR′— linking moiety, as these phrases are defined hereinabove, with R′ and R″ as defined herein.

The term “dithiocarbamate” as used herein encompasses N-dithiocarbamate and S-dithiocarbamate.

The term “S-dithiocarbamate” describes a —SC(═S)—NR′R″ end group or a —SC(═S)NR′— linking moiety, as these phrases are defined hereinabove, with R′ and R″ as defined herein.

The term “N-dithiocarbamate” describes an R″SC(═S)NR′— end group or a —SC(═S)NR′— linking moiety, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking moiety, as these phrases are defined hereinabove, where R′ and R″ are as defined herein and R″′ is as defined herein for R′ and R″.

The term “thiourea”, which is also referred to herein as “thioureido”, describes a —NR′—C(═S)—NR″R″′ end group or a —NR′—C(═S)—NR″— linking moiety, with R′, R″ and R″′ as defined herein.

The term “amide” as used herein encompasses C-amide and N-amide.

The term “C-amide” describes a —C(═O)—NR′R″ end group or a —C(═O)—NR′— linking moiety, as these phrases are defined hereinabove, where R′ and R″ are as defined herein.

The term “N-amide” describes a R′C(═O)—NR″— end group or a R′C(═O)—N— linking moiety, as these phrases are defined hereinabove, where R′ and R″ are as defined herein.

The term “imine”, which is also referred to in the art interchangeably as “Schiff-base”, describes a —N═CR′— linking moiety, with R′ as defined herein or hydrogen. As is well known in the art, Schiff bases are typically formed by reacting an aldehyde or a ketone and an amine-containing moiety such as amine, hydrazine, hydrazide and the like, as these terms are defined herein. The term “aldimine” refers to a —CH═N— imine which is derived from an aldehyde. The term “ketimine” refers to a —CR′═N— imine which is derived from a ketone.

The term “hydrazone” refers to a —R′C═N—NR″— linking moiety, wherein R′ and R″ are as defined herein.

The term “semicarbazone” refers to a linking moiety which forms in a condensation reaction between an aldehyde or ketone and semicarbazide. A semicarbazone linking moiety stemming from a ketone is a —R′C═NNR″C(═O)NR″′—, and a linking moiety stemming from an aldehyde is a —CR′═NNR″C(═O)NR″′—, wherein R′ and R″ are as defined herein and R″′ or as defined for R′.

As used herein, the term “lactone” refers to a cyclic ester, namely the intra-condensation product of an alcohol group —OH and a carboxylic acid group —COOH in the same molecule.

As used herein, the term “lactam” refers to a cyclic amide, as this term is defined herein. A lactam with two carbon atoms beside the carbonyl and four ring atoms in total is referred to as a β-lactam, a lactam with three carbon atoms beside the carbonyl and five ring atoms in total is referred to as a γ-lactam, a lactam with four carbon atoms beside the carbonyl and six ring atoms in total is referred to as a δ-lactam, and so on.

The term “guanyl” describes a R′R″NC(═N)— end group or a —R′NC(═N)— linking moiety, as these phrases are defined hereinabove, where R′ and R″ are as defined herein.

The term “guanidine” describes a —R′NC(═N)—NR″R′″ end group or a —R′NC(═N)—NR″— linking moiety, as these phrases are defined hereinabove, where R′, R″ and R″′ are as defined herein.

The term “hydrazine” describes a —NR′—NR″R″′ end group or a —NR′—NR″— linking moiety, as these phrases are defined hereinabove, with R′, R″, and R″′ as defined herein.

As used herein, the term “hydrazide” describes a —C(═O)—NR′—NR″R″′ end group or a —C(═O)—NR′—NR″— linking moiety, as these phrases are defined hereinabove, where R′, R″ and R′″ are as defined herein.

The term “hydroxylamine”, as used herein, refers to either a —NHOH group or a —ONH₂.

As used herein, the terms “azo” or “diazo” describe a —N═N—R′ end group or a —N═N— linking moiety, as these phrases are defined hereinabove, where R′ is as defined herein.

As used herein, the term “azido” described a —N═N⁺═N⁻ (—N₃) end group.

The term “triazine” refers to a heterocyclic ring, analogous to the six-membered benzene ring but with three carbons replaced by nitrogen atoms. The three isomers of triazine are distinguished from each other by the positions of their nitrogen atoms, and are referred to as 1,2,3-triazine, 1,2,4-triazine, and 1,3,5-triazine. Other aromatic nitrogen heterocycles include pyridines with 1 ring nitrogen atom, diazines with 2 nitrogen atoms in the ring and tetrazines with 4 ring nitrogen atoms.

The term “triazole” refers to either one of a pair of isomeric chemical compounds with molecular formula C₂H₃N₃, having a five-membered ring of two carbon atoms and three nitrogen atoms, namely 1,2,3-triazoles and 1,2,4-triazoles.

The term “aziridine”, as used herein, refers to a reactive group which is a three membered heterocycle with one amine group and two methylene groups, having a molecular formula of —C₂H₃NH.

As used herein, the term “thiohydrazide” describes a —C(═S)—NR′—NR″R″′ end group or a —C(═S)—NR′—NR″— linking moiety, as these phrases are defined hereinabove, where R′, R″ and R′″ are as defined herein.

As used herein, the term “methyleneamine” describes an —NR′—CH₂—CH═CR″R′″ end group or a —NR′—CH₂—CH═CR″— linking moiety, as these phrases are defined hereinabove, where R′, R″ and R′″ are as defined herein.

The term “diene”, as used herein, refers to a —CR′═CR″—CR″′═CR″″— group, wherein R′ as defined hereinabove, and R″, R″′ and R″″ are as defined for R′.

The term “dienophile”, as used herein, refers to a reactive group that reacts with a diene, typically in a Diels-Alder reaction mechanism, hence a dienophile is typically a double bond or an alkenyl.

The term “epoxy”, as used herein, refers to a reactive group which is a three membered heterocycle with one oxygen and two methylene groups, having a molecular formula of —C₂H₃O.

The phrase “covalent bond”, as used herein, refers to one or more pairs of electrons that are shared between atoms in a form of chemical bonding.

According to some embodiments of the present invention, some linking moieties result from a reaction between two reactive groups. Alternatively, a desired linking moiety is first generated and a bioactive agent and/or a spacer moiety are attached thereto.

### Linking Moiety Lability

A linking moiety that is stable at physiological conditions, namely the linking moiety does not disintegrate for the duration of exposure to the physiological environment in the bodily site, is referred to herein a “biostable linking moiety”. An exemplary biostable linking moiety is a triazole-based linking moiety. It is noted that biostability is also a relative term, meaning that a biostable linking moiety takes longer to break or requires certain cleavage conditions which hare less frequently encountered by the molecular structure when present in physiological conditions.

In some embodiments of the present invention, the linking moieties in the molecular structure provided herein are not biocleavable linking moieties.

In some embodiments of the present invention, the linking moieties in the molecular structure provided herein are all biocleavable. In the context of some embodiments of the present invention, biocleavable linking moieties are selected so as to break and release the bioactive agent attached thereto at certain conditions, referred to herein as “drug-releasing conditions” or “biocleavage conditions”.

According to some embodiments of the present invention, some of the linking moieties are biocleavable-linking moieties. As used herein, the terms “biocleavable” and “biodegradable” are used interchangeably to refer to moieties that degrade (i.e., break and/or lose at least some of their covalent structure) under physiological or endosomal conditions. Biodegradable moieties are not necessarily hydrolytically degradable and may require enzymatic action to degrade.

As used herein, the terms “biocleavable moiety” or “biodegradable moiety” describe a chemical moiety, which undergoes cleavage in a biological system such as, for example, the digestive system of an organism or a metabolic system in a living cell.

In some embodiments, biocleavable linking moieties are selected according to their susceptibility to certain enzymes that are likely to be present at the targeted bodily site or at any other bodily site where cleavage is intended, thereby defining the cleavage conditions.

Representative examples of biocleavable moieties include, without limitation, amides, carboxylates, carbamates, phosphates, hydrazides, thiohydrazides, disulfides, epoxides, peroxo and methyleneamines. Such moieties are typically subjected to enzymatic cleavages in a biological system, by enzymes such as, for example, hydrolases, amidases, kinases, peptidases, phospholipases, lipases, proteases, esterases, epoxide hydrolases, nitrilases, glycosidases and the like.

For example, hydrolases (EC number beginning with 3) catalyze hydrolysis of a chemical bond according to the general reaction scheme A-B+H₂O→A-OH+B-H. Ester bonds are cleaved by sub-group of hydrolases known as esterases (EC number beginning with 3.1), which include nucleases, phosphodiesterases, lipases and phosphatases. Hydrolases having an EC number beginning with 3.4 are peptidases, which act on peptide bonds.

Additional information pertaining to enzymes, enzymatic reactions, and enzyme-linking moiety correlations can be found in various publically accessible sources, such as Bairoch A., “The ENZYME database in 2000”, Nucleic Acids Res, 2000, 28, pp. 304-305.

In some embodiments, certain linking moieties are selected to be more labile, such as the L₁ L₂ . . . and Lₙ linking moieties, which are defined in general Formula I as the moieties linking between the various bioactive agents in the molecular structure presented herein. By “more labile”, it is meant that some of the linking moieties have a higher tendency to break at given cleavage conditions compared to other linking moieties. In some embodiments, the linking moieties are selected according to a certain lability hierarchy that allows the design of a particular drug-releasing profile, and/or a particular multi-drug-releasing profile, wherein the order and the rate of drug release is controllable according to the lability hierarchy. In the context of some embodiment of the invention, the more labile linking moieties, higher in the lability hierarchy will break first and at a higher rate than those lower in the lability hierarchy. The ability to select linking moieties according to their lability hierarchy provides molecular structures with differential multi-drug releasing profiles, according to some embodiments of the present invention.

The selection of the linking moieties according to lability hierarchy is determined according to the cleavage conditions, which the molecular structure is expected to experience once it is administered into a living cell/tissue/organ (collectively referred to herein as a “bodily site”). Cleavage conditions include the chemical and physical conditions that are present in the bodily site, such as temperature, pH, the presence of reactive species and the presence of enzymes, all of which can cause a given linking moiety to break and release the bioactive agent attached thereto.

For example, some linking moieties are more labile (susceptible to) in higher temperatures, while others are susceptible to higher or lower pH values compared to other linking moieties. In such cases, a molecular structure which is design to target a bodily site that is characterized by a localized pH value compared to its surroundings, an acid-labile or an H⁺-labile linking moiety is advantageously selected to release the bioactive agent it bears.

In some embodiments, the linking moieties in the string are selected so as to release the bioactive agent at the end of the string, and so on, namely the biocleavable linking moieties are selected to exhibit a gradient of biostability going from high to low starting from L₁ and on to L₂, . . . Lₙ and Lₙ₊₁. In such embodiments, D₁, D₂ . . . and Dₙ are released at a predetermined and controlled order starting from the last bioactive agent of the string (Dₙ) to the first bioactive agent attached to the HA moiety (D₁).

In some embodiments, each of the linking moieties is characterized by a given cleavage condition, and any one of the linking moieties is selected such that at least one thereof is different than one-another, based on the cleavage condition thereof. In some embodiments, each of the linking moieties is selected such that it is characterized by having a different cleavage condition, such that L₁ is more biostable than L₂, L₂ is more biostable than L₃, and so on to Lₙ₋₁ more biostable than Lₙ.

### Applications

Since the molecular structures presented herein carry, deliver and controllably release a wide variety of drugs, the molecular structures can be used to treat various medical conditions, and in particular, dermatologic conditions. The molecular structures presented herein can therefore be used as an active ingredient in a variety of pharmaceutical and cosmetic compositions, and in the preparation of a variety of medicaments.

Accordingly there is provided a pharmaceutical composition and/or a cosmetic composition that includes, as an active ingredient, the molecular structure provided herein, according to embodiments of the present invention, and a pharmaceutically and/or cosmetically acceptable carrier.

Similarly, there is provided a use of the molecular structure, according to embodiments of the present invention, in the preparation of a pharmaceutical and/or a cosmetic medicament.

According to some embodiments of the present invention, the pharmaceutical composition or medicament, are used to treat a medical or a dermatologic or a cosmetic condition, and more preferably a dermatologic/skin condition.

Also provided herein is a method of treating a skin condition in a subject in need thereof, which includes administering to the subject an effective amount of the molecular structure, according to embodiments of the present invention.

As used herein, the phrase “effective amount” describes an amount of a bioactive agent or a molecular structure being administered, which will relieve to some extent one or more of the symptoms of the dermatologic/skin condition being treated. In the context of the present embodiments, the phrase “effective amount” describes an amount of a molecular structure being administered and/or re-administered, which will relieve to some extent one or more of the symptoms of the dermatologic/skin condition being treated by being at a level that is beneficial to the target cell(s) or tissue(s), and effects a notable betterment of the skin condition.

In the context of embodiments of the present invention, the effective amount may refer to the molecular structure as a whole or to the amount of one or more bioactive agent releasably attached thereto. The efficacy of any bioactive agent, including the molecular structures presented herein, can be determined by several methodologies known in the art.

According to another aspect of embodiments of the present invention, any one of the molecular structures described herein is identified for use in treating a subject diagnosed with a skin condition treatable by at least one of the bioactive agents linked and controllably releasable from the molecular structure.

According to another aspect of embodiments of the present invention, there is provided a use of any one of the molecular structures described herein as a medicament. In some embodiments, the medicament is for treating a subject diagnosed with a skin condition treatable by at least one of the bioactive agents linked and controllably releasable from the molecular structure.

In any one of the methods and uses described herein, the molecular structure can be administered as a part of a pharmaceutical or cosmetic composition, which further comprises a pharmaceutically and/or cosmetically acceptable carrier, as known in the art. The carrier is selected suitable to the selected route of administration.

The molecular structures presented herein can be administered via several administration route, including, but not limited to, topically, subcutaneous, and orally. In some preferred embodiments, the molecular structure provided herein is administered using percutaneous and minimally invasive tools and methods, typically used to treat numerous dermatologic/skin conditions.

As a composition for treating various skin conditions, the molecular structure is particularly useful for topical and/or subcutaneous administration. In some embodiments, the preferred mode of administration is microneedling, also known as collagen induction therapy, which is a process involving repetitive and shallow puncturing of the skin with sterilized microneedles. Microneedling is typically effected by use of a dermaroller, whereas the cosmetic composition that includes the presently disclosed molecular structure, is applied on the skin area to be treated, and the dermaroller is used over this skin area. Alternatively, a dermaroller can be laced or loaded with the composition of the molecular structure. Further alternatively, microneedling for introduction of the molecular structure presented herein is effected by a syringe equipped with a small subcutaneous needle for shallow (2-3 mm) skin penetration.

According to some embodiments of the present invention, the molecular structure can be co-administered with one or more known drugs, compositions, medicaments and drugs suitable for treating a dermatologic/skin condition.

According to some embodiments, the composition comprising the molecular structure provided herein is packaged in a packaging material and identified in print, in or on the packaging material, for use in the treatment of a dermatologic/skin condition treatable by at least one of the entities linked and controllably releasable from the molecular structure, including the drugs and HA.

As used herein the term “composition” or the term “medicament” refer to a preparation of the molecular structures presented herein, with other chemical components such as pharmaceutically/dermatologically/cosmetically acceptable and suitable carriers and excipients, and optionally with additional bioactive agents or compositions comprising the same. The purpose of a pharmaceutical or cosmetic composition is to facilitate administration of the molecular structure to a subject.

Hereinafter, the phrase “pharmaceutically and/or cosmetically acceptable carrier” refers to a carrier or a diluent that does not cause significant irritation to the subject of the treatment and does not abrogate the biological activity and properties of the administered molecular structure. Examples, without limitations, of pharmaceutically and/or cosmetically acceptable carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a molecular structure. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, PA, latest edition, which is incorporated herein by reference.

The dosage may vary depending upon the dosage form employed and the route of administration utilized. The exact formulation, route of administration and dosage can be chosen by the individual caretaker in view of the subject's condition. (See e.g., Fingl et al., 1975, in “The Pharmacological Basis of Therapeutics”, Ch. 1 p.1). In general, the dosage is related to the efficacy of the active ingredient and the severity of the skin condition. The amount of a composition to be administered will, of course, be dependent on the subject being treated, the severity of the affliction, the manner of administration, the judgment of the prescribing caretaker, etc.

### Medical Conditions

The molecular structure presented herein can be used to treat dermatologic/skin conditions that are treatable by administration of a bioactive agent (drug) that is releasable form therefrom. Dermatologic/skin conditions can be caused by environmental factors, age and genetic factors, cancer, autoimmunity, and microorganisms.

Skin diseases and conditions, including nail and hair, are caused by viruses, rickettsiae, bacteria, fungi, and parasites. In some embodiments of the present invention, the skin condition treatable by the molecular structure provided herein is associated with an infection caused by a microorganism, including a viral infection, a bacterial infection, a yeast infection, a fungal infection, a protozoan infection, a parasite-related infection and the like.

Skin/dermatological conditions associated with a microorganism include, without limitation, impetigo, cellulitis and erysipelas, staphylococcal scalded skin syndrome, folliculitis, erysipeloid, pitted keratolysis, erythrasma, trichomycosis, intertrigo, acute infectious eczematoid dermatitis, pseudofolliculitis of the beard, toe web infection, skin tuberculosis (localized form), Mycobacterium marinum skin disease, Mycobacterium ulcerans skin disease, actinomycetoma, and actinomycosis.

In some embodiments, the molecular structure is design to release bioactive agents that are beneficial in the treatment of skin conditions, such as, but not limited to, melasma, skin whitening, hyperpigmentation, Chadwick's sign, Linea alba, Perineal raphe, acne scarring, acne, liver spots, surgical scars, stretch marks, and hair loss.

### Preparation of Molecular Structures

As can be appreciated form the diversity and complexity of the scope of embodiments of the present invention, the molecular structure provided herein can be afforded via various synthetic approaches. For example, the construction of the molecular structure can start with attaching one type of bioactive agent moiety to pristine HA strands, or HA strands that underwent modification of some of their functional groups, and continue with stepwise elongation and/or branching of the string of bioactive agent moieties, one bioactive agent moiety at a step. Alternatively, the process may start with forming a string of bioactive agent moieties, which is thereafter attached to a pristine or modified HA strand. The conjugation of two or more HA strands to for a crosslinked molecular structure can also be afforded by various synthetic approaches, which include linking a second HA strand to the end of a string on a first HA strand, or linking the ends of two strings on separate HA strands. In general, the molecular structure can also be afforded by any combination of the aforementioned synthetic approaches.

According to an aspect of some embodiments of the present invention, there is provided a process of preparing the molecular structure presented herein, which includes the following basic steps:
i) linking a first bioactive agent (D₁) to a functional group on a HA moiety via a first biocleavable linking moiety (L₁); and
ii) linking a second bioactive agent (D₂) to a functional group on the first bioactive agent moiety (D₁), thereby forming a second linking moiety (L₂).

The process can stop here, forming a molecular structure having 2 releasable bioactive agents (n=2), or continue to elongating the string by repeating step (ii) to the n^{th} bioactive agent moiety:
iii) after n−1 times, linking the last bioactive agent (Dₙ) to a functional group on the n−1 bioactive agent moiety (Dₙ₋₁), thereby forming the n^{th} linking moiety (Lₙ).

In some embodiments, the process starts with an optional step of modifying a native functional group on the HA strand such that the HA exhibits a modified functional group. In such embodiments, modification of functional groups in the HA strand facilitates the synthesis and the sequential attachment of bioactive agent moieties in the string-elongation steps. Thereafter, the process continues with steps (i)-(ii) or steps (i)-(iii) as presented hereinabove. The optional step includes reacting HA with one or more functional group modificators, as exemplified hereinabove, to afford a functional group modification moiety to which bioactive agent(s) or strings thereof, are attached.

It is noted herein that the construction of a molecular structure, according to embodiments of the present invention, can follow a different sequence of assembly steps, such as the construction of the fully formed string of releasable bioactive agents linked to one-another by biocleavable linkers starting or followed by linking thereof to the HA moiety, or the simultaneous attachment of identical bioactive agents to a fully formed linear and/or branched/dendrimeric string, or the attachment of fully formed stings to a the HA moiety and so on.

The process presented hereinabove is also useful for affording a crosslinked molecular structure, as follows:

A second HA moiety (HA₂) can be attached to the end of a string, regarded as the last bioactive agent moiety (Dₙ=HA₂); or

A first bioactive agent moiety at the end of a first string (a first Dₙ) attached to a first HA moiety (HA₁), is reacted with a second bioactive agent moiety at the end of a second string (a second Dₙ) attached to a second HA moiety (HA₂) such that a biocleavable linking moiety is formed from two reactive functional groups on each of the bioactive agent moieties. Such a synthetic approach forms molecular structures having crosslinking strings that connect two or more HA moieties.

According to some embodiments, some or all the steps of linking the various components of the molecular structure to one another further includes attaching controlled and sequential removal a variety of protection groups on the various functional groups. It is noted herein that similar protecting groups can be used to render one or more of the bioactive agents prodrugs/precursors of the same, once the protecting group is cleaved-off the bioactive agent; this practice is particularly useful in cases of bioactive agents that have more than two reactive functional groups, which need to be protected during the string elongation process.

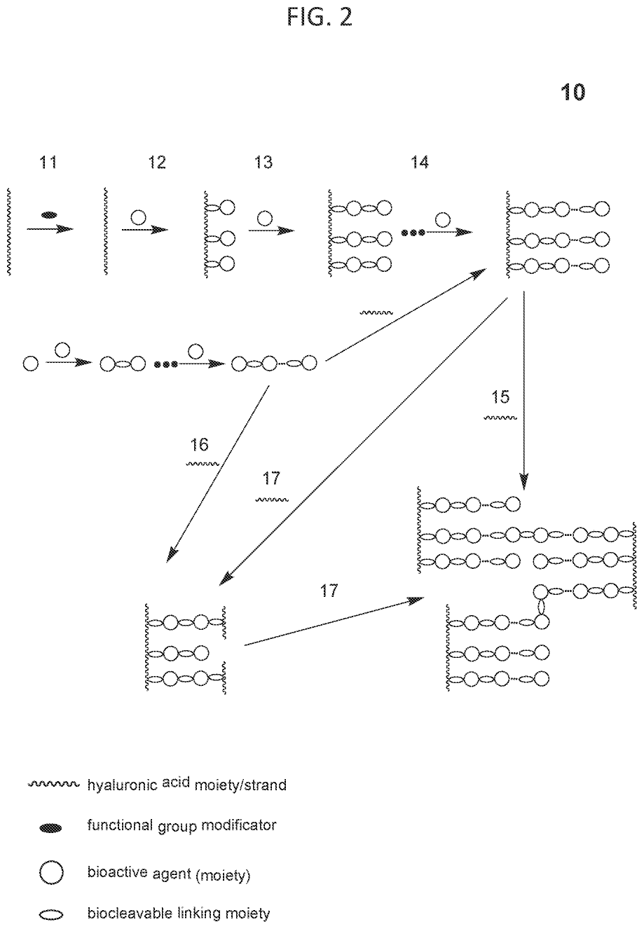

FIG. 2 presents a schematic representation of steps in several processes for preparing the molecular structure provided herein (10), according to some embodiments of the present invention, wherein Step 11 shows an optional modification of a hyaluronic acid strand by a functional group modificator that adds a functional group modification moiety to the HA moiety, Step 12 shows the attachment of the first bioactive agent moiety via a first biocleavable linking moiety to the HA moiety optionally having a modified functional group, Step 13 shows the attachment of a second bioactive agent moiety via a second biocleavable linking moiety to the first bioactive agent moiety, Step 14 shows the optional elongation of the string with additional bioactive agent moieties, Step 15 the optional crosslinking of at least two molecular structures via a biocleavable linking moiety to form a crosslinking string, and Steps 16 and Step 17 shows the optional attachment of a second hyaluronic acid moiety to some of the strings of a molecular structure thereby forming a crosslinked molecular structure.

As used herein, the term “protecting group” or “suitable protecting group”, refers to amino protecting groups, hydroxyl protecting groups and the like, depending on its location within the compound and includes those described in detail in Protecting Groups in Organic Synthesis, T. W. Greene and P. G. M. Wuts, 3^{rd} edition, John Wiley & Sons, 1999.

Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

Each of the abovementioned protecting groups can be used in the process of preparing the molecular structure provided herein, and also be used to render any one of the bioactive agents a prodrug of the same.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

When applied to an original property, or a desired property, or an afforded property of an object or a composition, the term “substantially maintaining”, as used herein, means that the property has not change by more than 20%, 10% or more than 5% in the processed object or composition.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It is expected that during the life of a patent maturing from this application many relevant molecular structures will be developed and the scope of the phrase “molecular structure” is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non-limiting fashion.

### Example 1

### Synthesis of Exemplary Molecular Structures

Following is an exemplary synthesis that demonstrates the preparation of an exemplary molecular structure, according to some embodiments of the present invention.

In this example, Boc protected tranexamic acid was first reacted with a cysteamine moiety S-(2-aminoethyl) ethanethioate using DCC, HOBt coupling method followed by Boc deprotection in 4N HCl in dioxane. The resulting S-(2-((1r,4r)-4-(aminomethyl)cyclohexanecarboxamido)ethyl) ethanethioate BU was coupled directly to HA via an amide linking moiety, thereby forming a string of two bioactive agents linked to HA. It is noted that although the language refers to a single string, multiple strings are formed simultaneously of multiple functionalities along the HA strand.

In this example, tranexamic acid—cysteamine dual bioactive agent moiety was couple to HA after forming a free amine functionality thereon, using HCl salt and washing with 1N NaHCO₃ solution prior to coupling.

Specifically, in this example, DMTMM (4-(4,6-dimethoxy-1,3,5-triazin-2-yl)-4-methyl-morpholinium chloride), an organic triazine derivative commonly used for act tranexamic acid moiety. The reaction was carried out for 48 hours in water with 5% AcCN, buffered to pH 6.3. The NMR spectra (not shown) indicated a high loading yield of 25-32%.

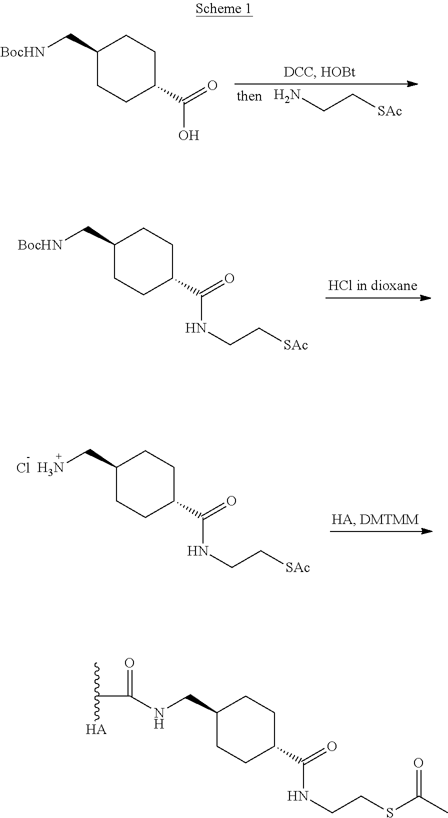

### Example 2

### An Exemplary Embodiment of a Molecular Structure

For a demonstration of a linear string of bioactive agents, Scheme 2A presents an exemplary embodiment of a molecular structure (Formula I), according to some embodiments of the present invention, comprising a hyaluronic acid residue, and a string of two tranexamic acid residues.

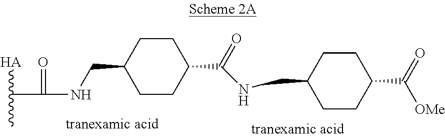

For another demonstration of a linear string of bioactive agents, Scheme 2B presents an exemplary embodiment of a molecular structure (Formula I), according to some embodiments of the present invention, comprising a hyaluronic acid residue, and a string of three tranexamic acid residues, wherein the end tranexamic acid residues is capped with a methyl ester protecting group.

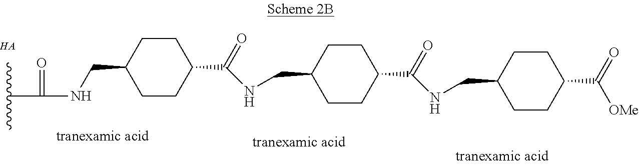

For a demonstration of a branched/dendrimeric string of bioactive agents, Scheme 2C presents an exemplary embodiment of a molecular structure (Formula I), according to some embodiments of the present invention, comprising a hyaluronic acid residue, and bis-tranexamic acid residues, wherein each of the tranexamic acid residues is capped with a methyl ester protecting group.

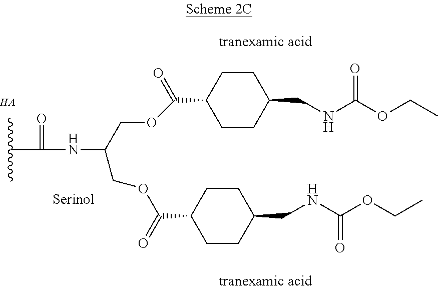 [SMILES: "HA" ; *C(=O)NC(COC(=O)[C@H]1CC[C@H](CNC(=O)OCC)CC1)COC(=O)[C@H]1CC[C@H](CNC(=O)OCC)CC1]

For another demonstration of a branched/dendrimeric string of bioactive agents, Scheme 2D presents an exemplary embodiment of a molecular structure (Formula I), according to some embodiments of the present invention, comprising a hyaluronic acid residue, and tris-tranexamic acid residues, wherein each of the tranexamic acid residues is capped with a ethyl carbamate protecting group.

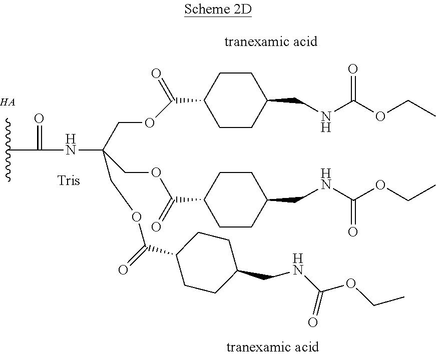

For yet another demonstration of a branched/dendrimeric string of bioactive agents, Scheme 2E presents an exemplary embodiment of a molecular structure (Formula I), according to some embodiments of the present invention, comprising a hyaluronic acid residue, an tranexamic acid residue and tris-tranexamic acid residues, wherein each of the tris-tranexamic acid residues is capped with a ethyl carbamate protecting group.

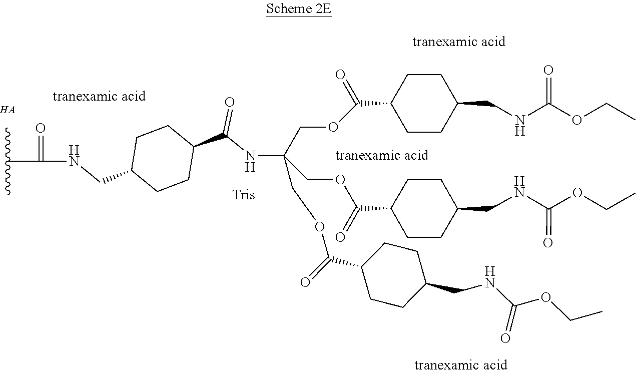

### Example 3

### An Exemplary Embodiment of a Molecular Structure

Scheme 3A presents an exemplary embodiment of a molecular structure (Formula I), according to some embodiments of the present invention, comprising a hyaluronic acid residue, a cysteamine residue, and a tranexamic acid residue, wherein PG is H or a protecting group such as acetamide, CO₂Me, Boc, SO₂Me, or semicarbazide.

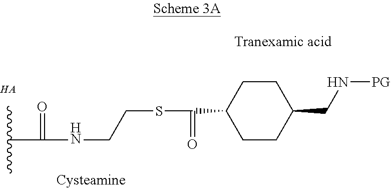

Scheme 3B presents an exemplary embodiment of a molecular structure (Formula I), according to some embodiments of the present invention, comprising a hyaluronic acid residue, a tranexamic acid residue, and a kojic acid residue.

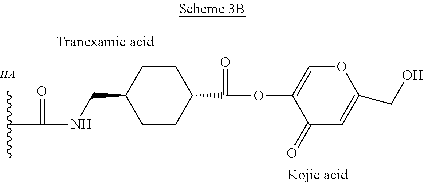

### Example 4

### An Exemplary Embodiment of a Molecular Structure

Scheme 4 presents an exemplary embodiment of a molecular structure (Formula I), according to some embodiments of the present invention, comprising a hyaluronic acid residue, tranexamic acid residue, and a kojic acid residue, wherein PG is H or a protecting group such as acetate, benzoate, nicotinade, and salicylate.

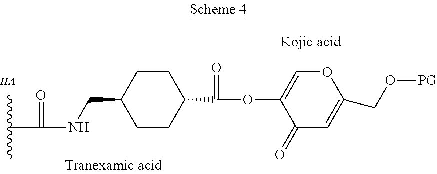

### Example 5

### An Exemplary Embodiment of a Molecular Structure

Scheme 5 presents an exemplary embodiment of a molecular structure (Formula I), according to some embodiments of the present invention, comprising a hyaluronic acid residue, a tranexamic acid residue, and an acetate-protected cysteamine residue.

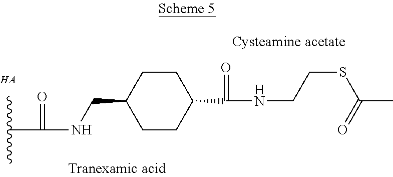

### Example 6

### An Exemplary Embodiment of a Molecular Structure

Scheme 6 presents an exemplary embodiment of a molecular structure (Formula I), according to some embodiments of the present invention, comprising a hyaluronic acid residue, a tranexamic acid residue, a kojic acid residue, and an acetyl-protected salicylic acid (acetsalicylate) residue.

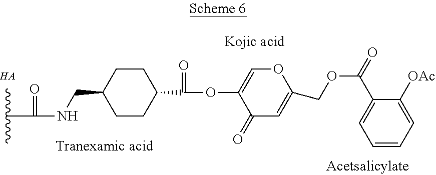

### Example 7

### An Exemplary Embodiment of a Molecular Structure

Scheme 7 presents an exemplary embodiment of a molecular structure, according to some embodiments of the present invention, comprising a hyaluronic acid residue, a tranexamic acid residue, a kojic acid residue, and a tretinoin residue.

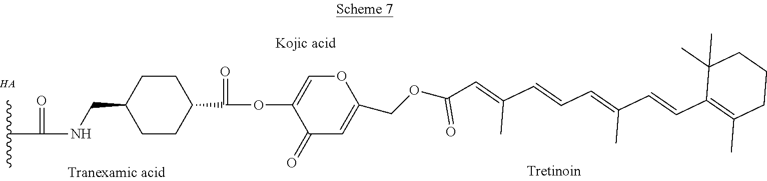

### Example 8

### An Exemplary Embodiment of a Molecular Structure

Scheme 8 presents an exemplary embodiment of a molecular structure, according to some embodiments of the present invention, comprising a hyaluronic acid residue, and a kojic acid residue between two tranexamic acid residues, wherein PG is H or a protecting group such as acetamide, CO₂Me, Boc, SO₂Me, or semicarbazide.

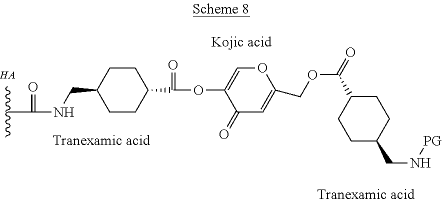

### Example 9

### An Exemplary Embodiment of a Molecular Structure

Scheme 9 presents an exemplary embodiment of a molecular structure, according to some embodiments of the present invention, comprising a hyaluronic acid residue, and a cysteamine residue between two tranexamic acid residues, wherein PG is H or a protecting group such as acetamide, CO₂Me, Boc, SO₂Me, or semicarbazide.

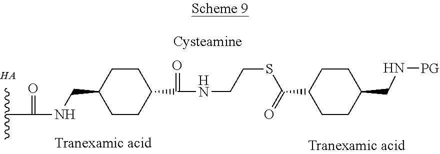

### Example 10

### An Exemplary Embodiment of a Molecular Structure

Scheme 10 presents an exemplary embodiment of a molecular structure, according to some embodiments of the present invention, comprising a hyaluronic acid residue, a tranexamic acid residue, a kojic acid residue, and a methyl ester-protected azelaic acid residue.

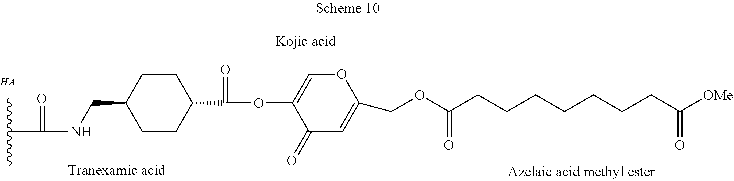

### Example 11

### An Exemplary Embodiment of a Molecular Structure

Scheme 11 presents an exemplary embodiment of a molecular structure, according to some embodiments of the present invention, comprising a hyaluronic acid residue, a tranexamic acid residue, a kojic acid residue, and a mequinol residue.

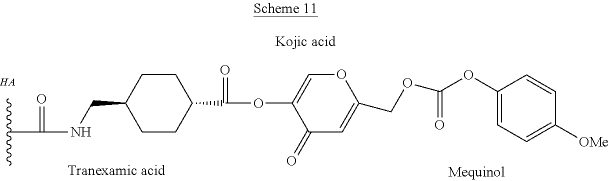

### Example 12

### An Exemplary Embodiment of a Molecular Structure

Scheme 12 presents an exemplary embodiment of a molecular structure, according to some embodiments of the present invention, comprising a hyaluronic acid residue, and a hydroquinone residue between two tranexamic acid residues, wherein PG is H or a protecting group such as acetamide, CO₂Me, Boc, SO₂Me, or semicarbazide.

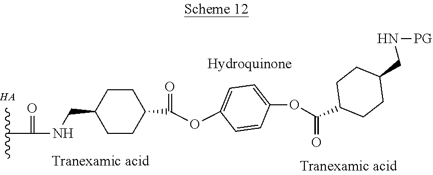

### Example 13

### An Exemplary Embodiment of a Molecular Structure

Scheme 13 presents an exemplary embodiment of a molecular structure, according to some embodiments of the present invention, comprising a hyaluronic acid residue, and a cystamine between two tranexamic acid residues, wherein PG is H or a protecting group such as acetamide, CO₂Me, Boc, SO₂Me, or semicarbazide.

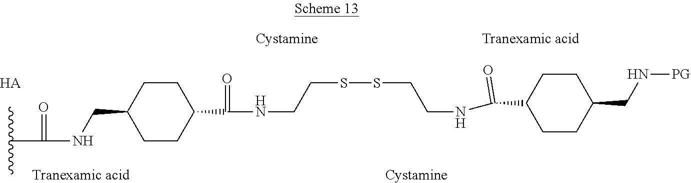

### Example 14

### An Exemplary Embodiment of a Molecular Structure

Scheme 14 presents an exemplary embodiment of a molecular structure, according to some embodiments of the present invention, comprising a hyaluronic acid residue, tranexamic acid residue, a cystamine residue, and an acetate-protected kojic acid residue.

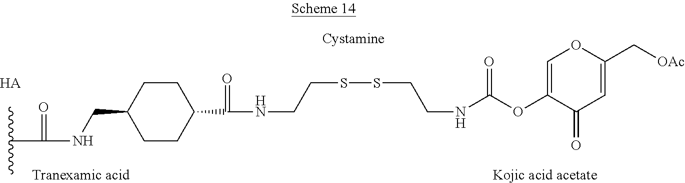

### Example 15

### An Exemplary Embodiment of a Molecular Structure

Scheme 15 presents an exemplary embodiment of a crosslinked molecular structure (Formula II), according to some embodiments of the present invention.

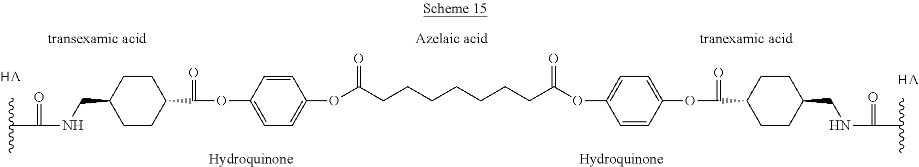

### Example 16

### An Exemplary Embodiment of a Molecular Structure

Schemes 16A-B present exemplary embodiments of crosslinked molecular structures (Formula II), according to some embodiments of the present invention.

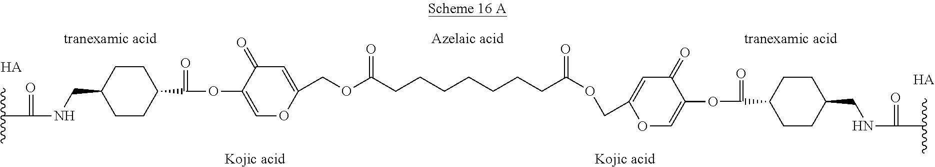

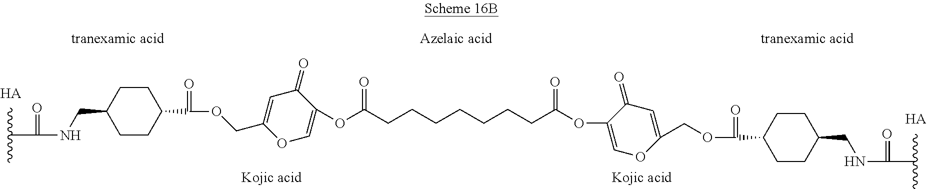

### Example 17

### An Exemplary Embodiment of a Molecular Structure

Schemes 17A-B present exemplary embodiments of crosslinked molecular structures (Formula II), according to some embodiments of the present invention.

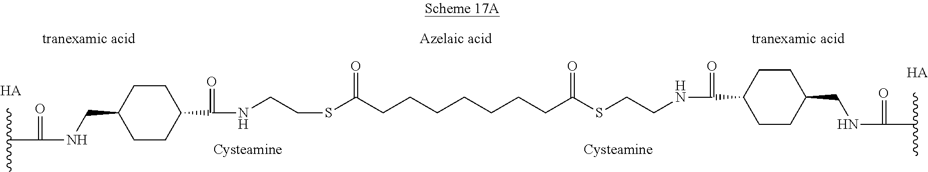

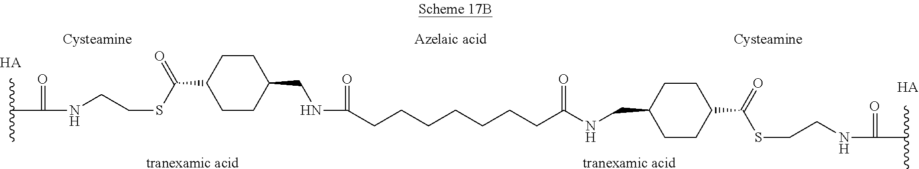

### Example 18

### An Exemplary Embodiment of a Molecular Structure

Schemes 18A-E present exemplary embodiments of crosslinked molecular structures (Formula II), according to some embodiments of the present invention.

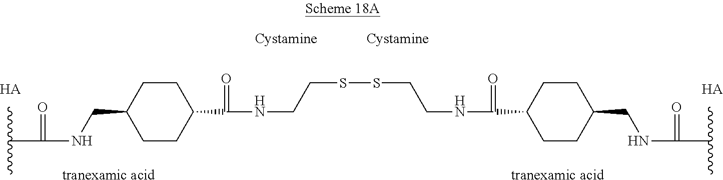

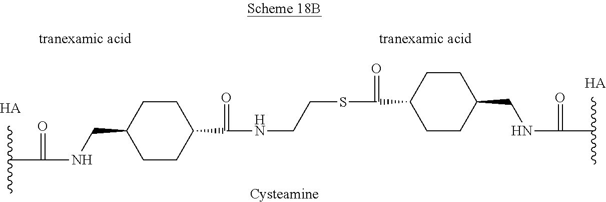

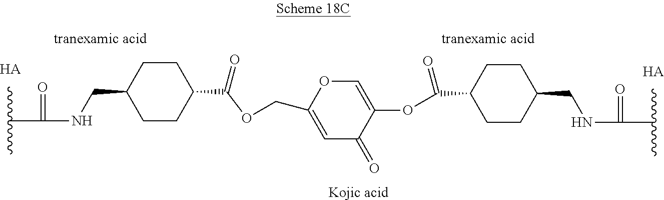

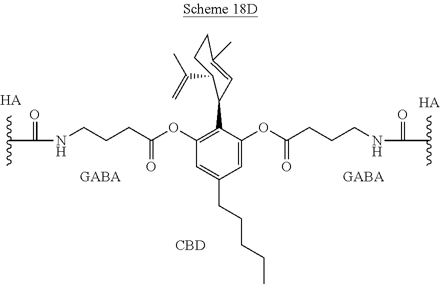

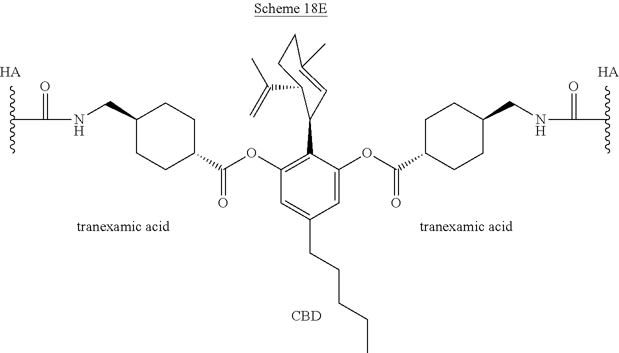

### Example 19

### An Exemplary Embodiment of a Molecular Structure

Schemes 19A-C present exemplary embodiments of crosslinked molecular structures (Formula II), wherein HA is modified to exhibit a functional group modification moiety, according to some embodiments of the present invention.

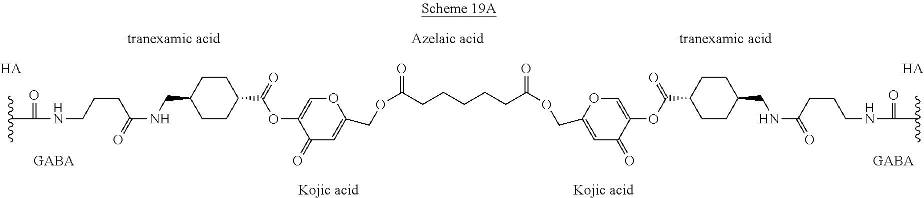

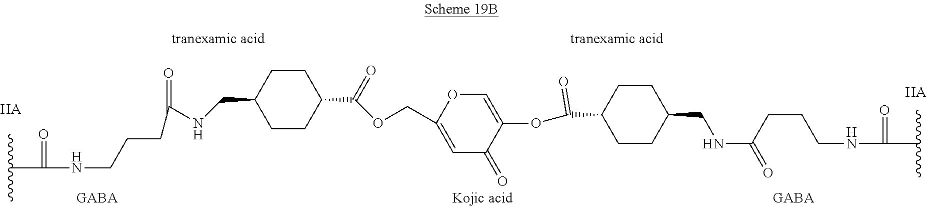

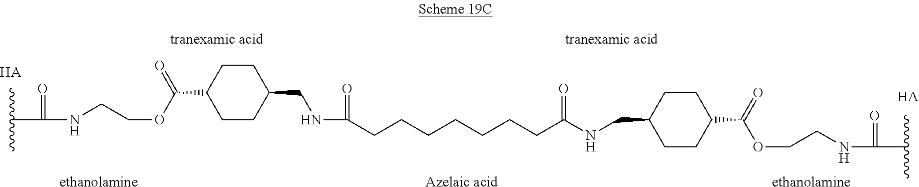

### Example 20

### Activity Assays

The molecular structures provided herein are tested for efficacy and safety according to an exemplary experimental protocol as follows.

Administration is effected by 0.2 mL subdermal injections using 30 G or 27 G hypodermic needles.

The dose frequency is 6 injection sites on the back of a rat administered once.

In-life study duration/handling is 13 weeks/14 weeks.

Morbidity and mortality: Twice a day.

Detailed clinical observation: prior to dosing, frequently for the first two hours post dosing, and twice weekly thereafter.

Grading for erythema and edema daily for the first 14 days, or until disappearance of erythema and edema (see table below).

Injection site measurement by caliper starting a day after injection and twice weekly thereafter.

A photograph of the back of each animal, right after injection and at 1, 2 and 3 months (before termination) after injection. A hair removal cream will be used before each photograph session.

Body weight monitoring: during acclimation and twice a week thereafter.

Necropsy and gross pathology: macroscopic findings on all main study animals at termination with special attention to draining lymph nodes.

Following termination, each animal receives one subdermal injection of 0.2 mL of a control or test article. The injected site is immediately sampled and fixed. These samples will allow the structural observation and histopathologic comparison with the injection sites sampled 13 weeks after injection.

Organ weight monitoring: any tissue with abnormal findings.

Tissue preservation: injection sites and any tissue with abnormal findings.

Histology/Pathology: Injection site will stained with, masson trichrome, hematoxylin eosin, and alcian blue (5 slides).

Grading system for histology: The qualitative and semi-quantitative evaluation of the histologic slides is conducted according to the ISO 10993.

For each test and control product, the mean irritation score is calculated among the 4 injection sites, each day.

Grading system for histologic evaluation (cell type/Response): polymorphonuclear cells, lymphocytes, plasma cells, macrophages, giant cells, necrosis, neovascularization, fibrosis, fatty infiltrate, fibrin, hemorrhage, fibroplasia, tissue integration, tissue ingrowth, encapsulation, product degradation.

Irritant Ranking Score (IRS, Table, Determination of IRS; ISO 10993): the individual irritation scores of the test and control products is calculated based on the histologic gradings as the sum F.1 of the tissue damage and cellular inflammatory parameter scores weighted with a factor 2 plus the sum F.2 of the repair phase of inflammation and fatty infiltrate parameter scores. The average individual irritation score (group average) is calculated as the mean result of the 3 injection sites per tested product. The IRS is determined by subtracting the control product (C1) average score to the test product average score. The IRS calculation is rounded to the nearest 0.1. A negative difference is recorded as zero.

The IRS is graded as follows:
non-irritant (0.0 to 2.9)
slight irritant (3.0 to 8.9)
moderate irritant (9.0 to 15.0)
severe irritant (=or >15.1).

TABLE 1

| Product (T1, T2, T3, C1 or C2) | | |
|---|---|---|
| Site 1 | Site 2 | Site 3 |

F.1 Inflammation
Polymorphonuclear cells
Lymphocytes
Plasma cells
Macrophages
Giant cells
Necrosis
F.1 SUBTOTAL ×2
F.2 Neovascularization
Neovascularization
Fibrosis
Fatty infiltrate
F.2 SUB TOTAL
TOTAL F.1 + F.2
GROUP AVERAGE

| | |
|---|---|
| IRS vs control C1 | IRS = Group average − Group average for C1 |
| IRS vs control C2 | IRS = Group average − Group average for C2 |

### Example 21

### Safety Studies

Pigmentary disorders in dermatology, such as lentigines, post-inflammatory hyperpigmentation and melasma, are not adequately treated at the present time. Lentigines or age spots are of universal occurrence in Caucasians due to the general aging process and exposure to sunlight. Melasma, which is characterized by blotchy, brown hyperpigmentation of the face, occurs in a high percentage of women on oral contraceptives. Post-inflammatory hyperpigmentation can accompany many skin diseases including chronic eczema, lichen planus and psoriasis. While some of these lesions can be treated with cryotherapy, alternative pharmacological approaches would be more readily accepted by both physicians and patients.

Several active agents are used to treat melisma, and include hydroquinone, tanexamic acid, kojic acid, cysteamine, azelaic acid, arbutin and the likes. These agents require long term use to produce depigmentation also because limited biostability in dermis.

The HA-based conjugates, according to some embodiments of the present invention, allow “slow” or controlled release of some of these agents, as well as improved modalities, increase half-life, and improved efficacy in treatment of melasma.

The safety of the herein disclosed HA-based molecular structures was tested as follows. One male brown pig age 6 months was housed in standard stainless steel pen at the Animal Facilities, Havat Keshet, Rehovot, and acclimated for at least two weeks prior to initiation of the study.

Prior to treatment, melanocytes were stimulated by UV irradiation following a published protocol [Nair, X. et al., Journal of Investigative Dermatology, 1993, 101(2), pp. 145-149]. A pattern of square areas, each 4 cm×4 cm in size and spaced 2 cm apart were chosen on the side of the animal for the treatment. Thereafter, the tested samples were dissolved in water/glycerol (2:1), and applied on the squares using a microneedling (mesotherapy) device at 2.5 mm depth. The treatment was delivered once a month for 3 months, affording three treatments in total.

The tested samples were:

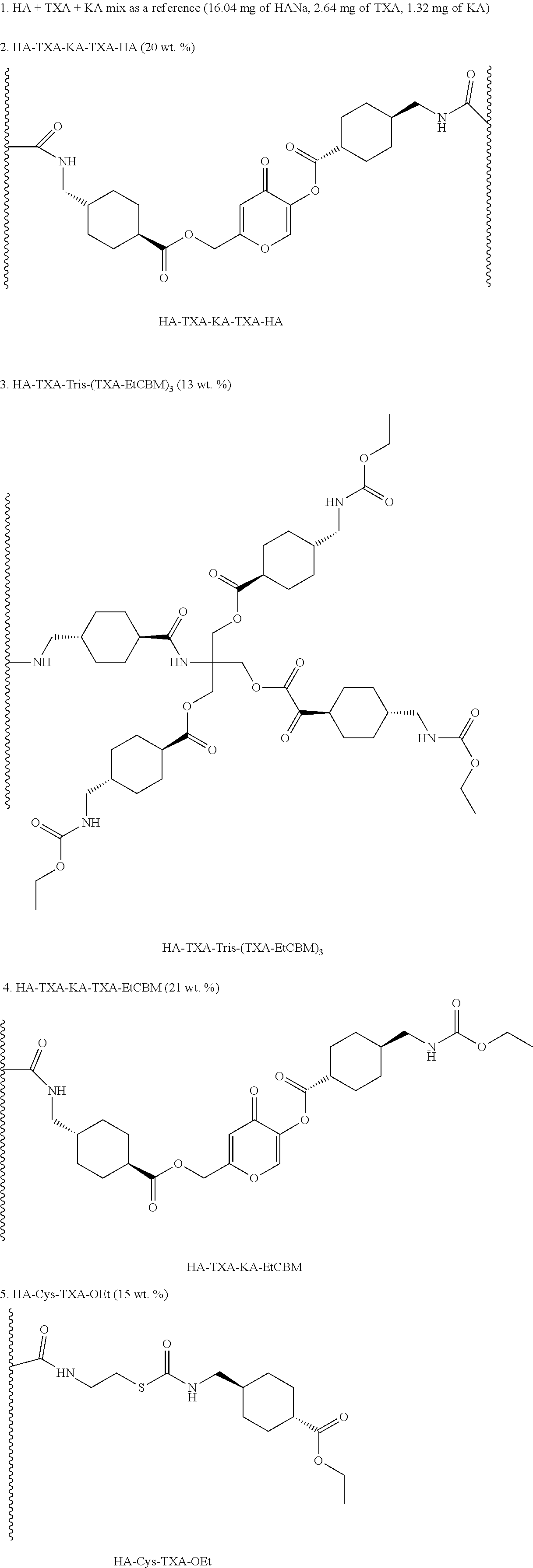

6. HA+Cys+TXA mix as a reference (10 mg of HANa, 5 mg of TXA, 5 mg of Cys*HCl)
7. HA+TXA mix as a reference (17.2 mg of HANa, 2.8 mg of TXA)
8. Untreated

At the end of experiment biopsies were taken and subjected to histopathology according to published protocols [Kerlin, R. et al., Toxicol Pathol., 2016, 44(2), pp. 147-62; Schafer, K. A. et al., Toxicol Pathol., 2018, 46(3), pp. 256-265].

### Organ/Tissue Collection and Fixation

Samples of skin (n=5) from one pig were harvested, fixed in 4% formaldehyde, and kept in the fixative for 48 hours for further fixation. The tissues were trimmed, put in embedding cassettes and processed routinely for paraffin embedding.

### Slide Preparation

Paraffin sections (4 microns thick) were cut, put on glass slides and stained with Hematoxylin & Eosin (H&E) for general histopathology, Masson Trichrome (MT) for collagen and Masson Fontana (MF) for melanin.

### Light Microscopy Photography

Pictures were taken, using Olympus microscope (BX60, serial NO. 7D04032) equipped with microscope's Camera (Olympus DP73, serial NO. OH05504) at objective magnification of ×10.

### Histopathological Evaluation

The H&E-stained slides were examined, described and scored by the study Pathologist, using a semi-quantitative grading scale, of 5-point scale, for the severity of the histopathological changes as follows:
Grade 0—The tissue appears normal.
Grade 1—Minimal pathological findings.
Grade 2—Mild pathological findings.
Grade 3—Moderate pathological findings.
Grade 4—Severe pathological findings.

The Masson Trichrome (MT) stained slides were examined and graded by a semi-quantitative scoring system for the presence of fibrosis/collagen (mag. ×10), as follows:
0—No signs of fibrosis/collagen degradation as appears in normal skin.
1—Mild fibrosis/collagen degradation.
2—Moderate fibrosis/collagen degradation.
3—Severe fibrosis/collagen degradation.

The Masson Fontana (MF) stained slides were examined and graded by a semi-quantitative scoring system for the presence of melanin (mag. ×10), as follows:
0—Almost no pigmented cells visible;
1—Few pigmented cells, less than in normal skin;
2—Normal number of pigmented cells, as compared to normal skin;
3—Increased number of pigmented cells;
4—High number of pigmented cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A molecular structure represented by Formula I:
HA [symbol] L₁-D₁-L₂-D₂ . . . Lₙ-Dₙ]ₘ Formula I
wherein:
HA is a first hyaluronic acid moiety;
L₁, L₂ . . . and Lₙ are each independently a biocleavable linking moiety;
D₁, D₂ . . . and Dₙ are each independently a bioactive agent moiety;
n is an integer equal or larger than 2; and
m is a positive number equal or larger than 1.

2. The structure of claim 1, wherein each of said D₁, D₂ . . . and Dₙ is a moiety of the same bioactive agent.

3. The structure of claim 1, wherein each of said D₁, D₂ . . . and Dₙ is a moiety of a different bioactive agent.

4. The structure of claim 1, wherein each of said L₁, L₂ . . . and Lₙ is the same biocleavable linking moiety.

5. The structure of claim 1, wherein each of said L₁, L₂ . . . and Lₙ is a different biocleavable linking moiety.

6. The structure of claim 5, wherein each of said L₁, L₂ . . . and Lₙ is characterized by a different biocleavage condition.

7. The structure of claim 1, further comprises a second hyaluronic acid moiety attached to the structure via Lₙ.

8. The structure of claim 7, represented by Formula II:
HA₁[-L₁-D₁-L₂-D₂ . . . Lₙ-Dₙ-Lₙ₊₁-HA₂]ₘ Formula II
wherein:
HA₁ is said first hyaluronic acid moiety; and
Lₙ₊₁ is a biocleavable linking moiety;
HA₂ is said second hyaluronic acid moiety.

9. The structure of claim 1, wherein said bioactive agent is selected from the group consisting of tranexamic acid, kojic acid, cysteamine, cystamine, azelaic acid, hydroquinone, mequinol, flutamide, CBD, arbutin, vitamin A, vitamin C, vitamin E, ellagic acid, glutathione, biotin, mandelic acid, an alpha-hydroxy acid, tretinoin, an alpha-lipoic acid, glutathione, salicylic acid and fluocinolone.

10. (canceled)

11. The structure of claim 1, wherein at least one functional group in said HA exhibits at least one functional group modification moiety.

12. The structure of claim 1, wherein at least one of D₁, D2 . . . and Dₙ is a precursor of a bioactive agent.

13. The structure of claim 1, represented by any one of the structures presented in Examples 1-19.

14. A cosmetic composition comprising the molecular structure of claim 1 as an active ingredient, and a cosmetically acceptable carrier.

15. The cosmetic composition of claim 14, being packaged in a packaging material and identified in print, in or on said packaging material, for use in the treatment of a skin condition.

16-17. (canceled)

18. A method of treating a skin condition in a subject in need thereof, the method comprising, administering to said subject an effective amount of the molecular structure of claim 1.

19. The method of claim 18, wherein said skin condition is selected from the group consisting of melasma, skin whitening, hyperpigmentation, Chadwick's sign, Linea alba, Perineal raphe, acne scarring, acne, liver spots, surgical scars, stretch marks and hair loss.

20. A process of preparing the molecular structure of claim 1, the process comprising:
i) attaching D₁ to HA via L₁;
ii) attaching D₂ to D₁ via L₂; and
iii) for n greater than 2, optionally attaching Dₙ to D₂ or to Dₙ₋₁ via L₂ or Dₙ; and/or
iv) attaching D₂ to D₁ via L₂;
v) for n greater than 2, optionally attaching Dₙ to D₂ or to Dₙ₋₁ via L₂ or Dₙ; and
vi) attaching D₁ to HA via L₁.

21. The process of claim 20, further comprising, prior to Step (i) and/or Step (vi), modifying at least one functional group in HA so as to exhibit at least one functional group modification moiety.

22. The process of claim 20, further comprising, attaching a second hyaluronic acid moiety to the molecular structure via Lₙ.

23. The process of claim 20, wherein at least one of D₁, D₂ . . . and Dₙ is a precursor of a bioactive agent.
